(19) United States
(12) Patent Application Publication
(10) Pub. No.: US 2024/0024572 A1
(43) Pub. Date: Jan. 25, 2024

(54) INFUSION SET WITH FEEDBACK MECHANISM

(71) Applicant: West Pharmaceutical Services, Inc., Exton, PA (US)

(72) Inventors: Ran Hezkiahu, Herzliya (IL); Tommy Gene Davis, Athens, TX (US); Ross Perry Vanstone, Scottsdale, AZ (US); Jason J. Fink, Tucson, AZ (US)

(21) Appl. No.: 18/022,989

(22) PCT Filed: Aug. 24, 2021
(86) PCT No.: PCT/US21/47369

§ 371 Date: Feb. 24, 2023

Related U.S. Application Data
(60) Provisional application No. 63/069,392, filed on Aug. 24, 2020.

(51) Int. Cl. A61M 5/158; A61M 5/172; A61M 5/32

(52) U.S. Cl. CPC A61M 5/158; A61M 5/1723; A61M 5/321; A61M 2205/3368; A61M 2205/3306

(57) ABSTRACT

An infusion set adapter connects to an infusion set for delivering a medicament to an injection site from a medicament source. The infusion set adapter can include a housing a controller, a power source, and at least one sensor configured to detect a characteristic of the infusion set adapter, infusion set, infusion site, and/or the medicament source. The controller can process information received from the one or more sensors and cause an action to occur in response to the received and processed information. The housing can include a retention member to connect the infusion set adapter to the infusion set. An infusion set include components of the infusion adapter above integrated therein. Methods of operating infusion sets and infusion set adapters are also disclosed.

### CROSS REFERENCE TO RELATED APPLICATIONS

This application claims the benefit of U.S. Provisional Application No. 63/069,392, filed Aug. 24, 2020, the entirety of which is incorporated herein.

### TECHNICAL FIELD

The disclosure generally relates to infusion sets connectable with infusion pumps, and, more particularly, to infusion sets employing a feedback mechanism.

### BACKGROUND

Infusion sets are generally utilized with infusion pumps to subcutaneously deliver a substance, e.g., medication from the pump to a patient. Conventional infusion sets include a needle or flexible cannula that is fluidly communicated with the substance within the infusion pump via a conduit. One drawback of conventional infusion sets, however, is that the infusion pump, which contains the majority of the operative components to dispense the substance, e.g., the medication, the drive system, the control system, and the like, cannot detect and does not receive any feedback from the infusion set. For example, the infusion pump, and ultimately the user, may be unaware whether the infusion set is properly connected with the infusion pump, whether the infusion set has been placed on the skin surface, whether the infusion set has been properly primed, whether the needle or cannula has been inserted into the skin and is positioned at a sufficient depth underneath the skin surface, and the like.

It would, therefore, be advantageous to manufacture an infusion set, or infusion set supplement, which communicates with the infusion pump and/or another patient interface device, i.e., provides feedback thereto, in order to provide patient notifications.

### SUMMARY

The foregoing needs are met by various aspects of infusion sets, infusion set adapters, and related methods disclosed. According to an aspect of this disclosure, an infusion set adapter is disclosed. The infusion set adapter can include a housing comprising a retention member configured to be connected to an infusion set, a controller and a power source configured to power the controller; and a sensor in electrical communication with the controller. The sensor can be selected from the group consisting of a priming sensor adjacent a cannula of the infusion set and configured to detect a presence of a medicament in the cannula or in another tube adjacent to the cannula, a proximity sensor configured to measure a characteristic of an infusion site, and a connection sensor configured to detect a characteristic indicative of a distance between the infusion set adapter and the infusion set.

The controller can be configured to cause a notification of the presence of the medicament in response to receiving a signal from the priming sensor. The power source can include a battery. The controller and the power source can be disposed on the housing. The controller can be disposed on the housing and the power source is disposed on a medicament source. The retention member can include a plurality of retention members configured to connect the housing to the infusion set. The retention member can be resilient and can be configured to deform to move over a retention section of the infusion set as the retention member is being connected to the infusion set. The infusion set adapter can include a needle configured to pierce into an infusion site.

The infusion set adapter can include a needle sensor configured to detect a characteristic of a needle tip of the needle within the infusion site. The needle sensor can be in electrical communication with the controller. The infusion set adapter can include a needle guard configured to cover a needle tip of the needle when the needle is not in the infusion site. The infusion set adapter can include a needle guard sensor configured to detect a characteristic of the needle guard relative to the needle tip. The needle guard sensor can be in electrical communication with the controller.

The sensor can be the proximity sensor. The controller can be configured to determine if the infusion set adapter is within a predetermined distance from the infusion site. The proximity sensor can include an optical sensor configured to transmit a first optical signal to the infusion site and to receive a second optical signal reflected from the infusion site. The proximity sensor can include a temperature sensor configured to measure a temperature adjacent the proximity sensor. The proximity sensor can include a mechanical sensor, an electromechanical sensor, or a pressure sensor configured to detect physical contact between the infusion set adapter and the infusion site.

The infusion set adapter can include the connection sensor on the housing. The connection sensor can be configured to detect a characteristic indicative of a distance between the infusion set adapter and the infusion set. The controller can be configured to determine if the infusion set adapter is within a predetermined distance from the infusion set.

The infusion set adapter can include a removable interface adapter connected to the housing. The retention member can be disposed on the interface adapter.

According to another aspect of this disclosure, an infusion set for delivering a medicament from a medicament source to an infusion site is disclosed. The infusion set can include a housing configured to be connected to the medicament source. The housing can have an attachment surface for contacting the infusion site. The infusion set can also include a cannula connected to the housing, where the cannula is configured to receive the medicament therein from the medicament source. The infusion set can also include a needle disposed on the housing, where the needle comprises a needle tip configured to pierce the infusion site, and a needle guard configured to cover the needle tip when the needle tip is not in the infusion site. The infusion set can also include a controller and a power source configured to provide power to the controller. The infusion set can also include a sensor in electrical communication with the controller, the sensor being selected from the group consisting of a priming sensor configured to detect a presence of the medicament in the cannula, a proximity sensor configured to measure a characteristic of the infusion site, a connection sensor configured to detect a characteristic indicative of a connection between the medicament source and the infusion set, a needle sensor configured to measure a characteristic of the needle tip, and a needle guard sensor configured to measure a characteristic of the needle guard.

The controller can be configured to cause a notification of the presence of the medicament in response to receiving a signal from the priming sensor. The priming sensor can be disposed within a lumen defined by the cannula or adjacent an outer surface of the cannula. The cannula can define a second lumen separated from the lumen. The controller and the power source can be disposed within the housing. The controller can be disposed on the housing, and the power source can be disposed on the medicament source. The medicament source can include a reservoir configured to receive the medicament therein and a pump configured to cause movement of the medicament from the reservoir to the infusion set. The infusion set can include adhesive on the attachment surface for securing the infusion set to the infusion site.

The sensor can be the proximity sensor, and the controller can be configured to determine if the infusion set is within a predetermined distance from the infusion site. The infusion set can include an actuator configured to move the needle from a sheathed position, in which the needle tip is spaced from the infusion site, to an unsheathed position, in which the needle tip contacts the infusion site. The needle can be configured to pierce the infusion site to create an aperture in the infusion site, and the cannula can be configured to be moved into the infusion site through the aperture.

According to another aspect of this disclosure, an infusion set for delivering a medicament from a medicament source to an infusion site is disclosed. The infusion set can include a body configured to attach to the infusion site, a needle extending from the body that is configured to pierce the infusion site, and a cannula connected to the body that receives the medicament from the medicament source therein. The infusion set can also include an infusion set adapter operably connected to the body. The infusion set adapter can have a controller, a power source configured to provide power to the controller, and a sensor in electrical communication with the controller. The sensor can be selected from the group consisting of a priming sensor disposed adjacent the cannula and configured to detect a presence of the medicament in the cannula, a proximity sensor configured to measure a characteristic of the infusion site, a connection sensor configured to detect a characteristic indicative of a distance between the infusion set adapter and the infusion set, and a needle sensor configured to measure a characteristic of the needle. In some aspects, the priming sensor can be configured to measure a parameter associated with the presence of air bubbles in the cannula and/or in adjacent tubing.

The infusion set adapter can be configured to be separated from the infusion set and sterilizable. The controller can be configured to cause a notification of the presence of the medicament in response to receiving a signal from the priming sensor. The priming sensor can be disposed within a lumen defined by the cannula or adjacent an outer surface of the cannula. The cannula can define a second lumen separated from the lumen.

The infusion set adapter can include a retention member configured to secure the infusion set adapter to the body of the infusion set. The retention member can be resilient and can be configured to deform to move over a retention section of the body of the infusion set as the infusion set adapter is being secured to the body of the infusion set. The connection sensor can be configured to detect a characteristic indicative of whether the infusion set adapter is secured to the body of the infusion set. The connection sensor can include an optical sensor, a mechanical sensor, an electromechanical sensor, or a pressure sensor configured to detect a characteristic indicative of relative position between the infusion set adapter and the body of the infusion set.

The needle sensor can be configured to detect a characteristic of a needle tip of the needle within the infusion site. The infusion set can include a needle guard configured to cover a needle tip of the needle when the needle is not in the infusion site. The infusion set can include an actuator configured to move the needle from a sheathed configuration, in which the needle tip is spaced from the infusion site, to an unsheathed configuration, in which the needle tip contacts the infusion site.

The proximity sensor can be configured to measure a characteristic of an infusion site. The controller can be configured to determine if the infusion set adapter is within a predetermined distance from the infusion site. The proximity sensor can include an optical sensor configured to transmit a first optical signal to the infusion site and to receive a second optical signal reflected from the infusion site. The proximity sensor can include a temperature sensor, a mechanical sensor, an electromechanical sensor, or a pressure sensor configured to detect a physical contact between the infusion set and the infusion site. The needle can be configured to pierce the infusion site to create an aperture in the infusion site, and the cannula can be configured to be moved into the infusion site through the aperture.

### BRIEF DESCRIPTION OF THE DRAWINGS

The present application is further understood when read in conjunction with the appended drawings. For the purpose of illustrating the subject matter, exemplary aspects of the subject matter are shown in the drawings; however, the presently disclosed subject matter is not limited to the specific methods, devices, and systems disclosed. In the drawings:

FIG. 1 illustrates an infusion set and a medicament source according to an aspect of this disclosure;

FIG. 2 illustrates a schematic diagram of a medicament source according to an aspect of this disclosure;

FIG. 3 illustrates a perspective view of an infusion set according to an aspect of this disclosure;

FIG. 4 illustrates another perspective view of the infusion set of FIG. 3;

FIG. 5 illustrates a perspective view of an infusion set adapter connected to an infusion set according to an aspect of this disclosure;

FIG. 6 illustrates a perspective view of the infusion set adapter of FIG. 5;

FIG. 7 illustrates another perspective view of the infusion set adapter of FIG. 5;

FIG. 8 illustrates yet another perspective view of the infusion set adapter of FIG. 5;

FIG. 9 illustrates a cross-sectional view of an infusion set adapter connected to an infusion set according to an aspect of this disclosure;

FIG. 10 illustrates a perspective view of a portion of the infusion set adapter of FIG. 5 attached to an infusion set according to an aspect of this disclosure;

FIG. 11 illustrates a perspective view of priming sensor adjacent an infusion set according to an aspect of this disclosure;

FIG. 12 illustrates a schematic diagram of an infusion set adapter according to an aspect of this disclosure;

FIG. 13A illustrates a perspective view of a cannula according to an aspect of this disclosure;

FIG. 13B illustrates a perspective view of a cannula according to another aspect of this disclosure;

FIG. 13C illustrates a perspective view of a cannula according to yet another aspect of this disclosure;

FIG. 13D illustrates a perspective view of a cannula according to yet another aspect of this disclosure;

FIG. 14 illustrates a flow chart depicting a method of operating an infusion set adapter according to an aspect of this disclosure;

FIG. 15 illustrates a perspective view of an infusion set according to another aspect of this disclosure;

FIG. 16 illustrates another perspective view of the infusion set of FIG. 15;

FIG. 17 illustrates a cross-sectional view of the infusion set of FIG. 15;

FIG. 18 illustrates an exploded assembly view of the infusion set of FIG. 15;

FIG. 19 illustrates an infusion set, infusion set adapter, and medicament source according to an aspect of this disclosure;

FIG. 20 illustrates a perspective view of the infusion set and infusion set adapter of FIG. 19, being shown in a disconnected configuration;

FIG. 21 illustrates the infusion set and infusion set adapter of FIG. 20 shown in a connected configuration;

FIG. 22 illustrates a perspective view of an infusion set adapter and a connected infusion set according to yet another aspect of this disclosure;

FIG. 23 illustrates another perspective view of the infusion set adapter and connection infusion set of FIG. 22;

FIG. 24 illustrates a cross-sectional view of the infusion set adapter of FIG. 22;

FIG. 25 illustrates yet another perspective view of a portion of the infusion set adapter of FIG. 22;

FIG. 26 illustrates a perspective view of a portion of an infusion set adapter according to yet another aspect of this disclosure;

FIG. 27 illustrates a bottom plan view of a portion of an infusion set adapter according to yet another aspect of this disclosure;

FIG. 28 illustrates a bottom plan view of a portion of an infusion set adapter according to yet another aspect of this disclosure;

FIG. 29 illustrates a bottom plan view of a portion of an infusion set adapter according to yet another aspect of this disclosure; and

FIG. 30 illustrates an exploded view of the portion of the infusion set adapter of FIG. 29.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

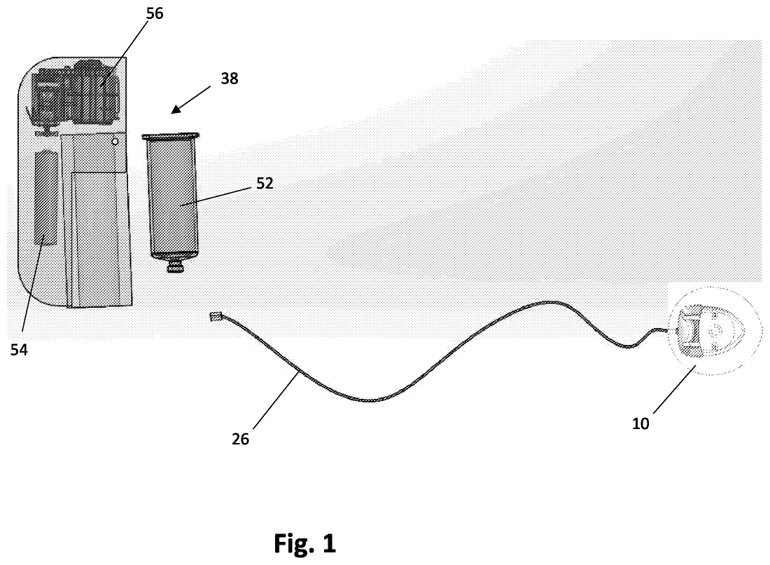

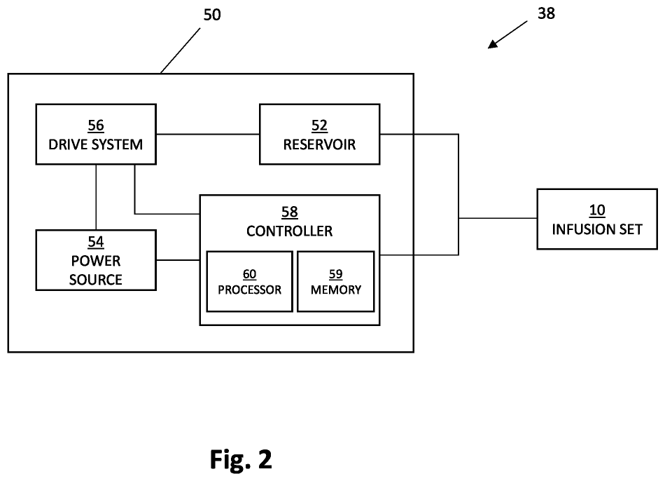

Referring to the drawings in detail, where like numerals indicate like elements throughout this application, FIGS. 1 and 2 depict an embodiment of an infusion set and a medicament source 38. Generally, the infusion set 10 can be used to transport one or more medicaments to a user. The infusion set 10 can be operably connected to the medicament source 38 and configured to receive the medicament from the medicament source 38. The infusion set 10 can then deliver the medicament to a user at an infusion site 40. The infusion site 40 can be on the patient's skin. In some cases, the infusion site 40 can include a separate infusion component that is attached to the patient's skin and that receives the medicament from the infusion set 10.

The medicament can be moved from the medicament source 38 by one or more known mechanisms, such as via pump, depression of a syringe, vacuum, capillary action, or another suitable mechanism. In some aspects, the medicament source 38 can include a pump mechanism configured to cause movement of the medicament into and through the infusion set 10. In some exemplary embodiments, as shown in FIGS. 1 and 2, the medicament source 38 can include an infusion pump 50 configured to receive the medicament therein and to move the medicament therefrom into and through the infusion set 10. The infusion pump 50 can include a reservoir 52 configured to receive the medicament therein. The reservoir 52 can include any suitable medicament container, such as a bottle, a vial, a syringe, or the like. The reservoir 52 may be a removable cartridge that is designed to be removed and/or replaced with respect to the rest of the infusion pump 50. The reservoir 52 may be attached to the infusion pump 50 prior to use.

The infusion pump 50 can include a power source 54, a drive system 56 (e.g., a pump), and a controller 58 (see schematic of FIG. 2). The power source 54 is configured to power the drive system 56 and the controller 58. The power source 54 may include a battery. The controller 58 can include a memory 59 and a processor 60, such as a microprocessor. The memory 59 can be configured to store computer code, software, instructions, data, or a combination thereof and the like for carrying out operation of the infusion pump 50. The infusion pump 50 may be operable in a manner understood by those of ordinary skill in the art. For example, the controller 58 can direct operation of the drive system 56 to discharge the medicament in the reservoir 52 into the infusion set 10 at a predetermined dosage and/or rate.

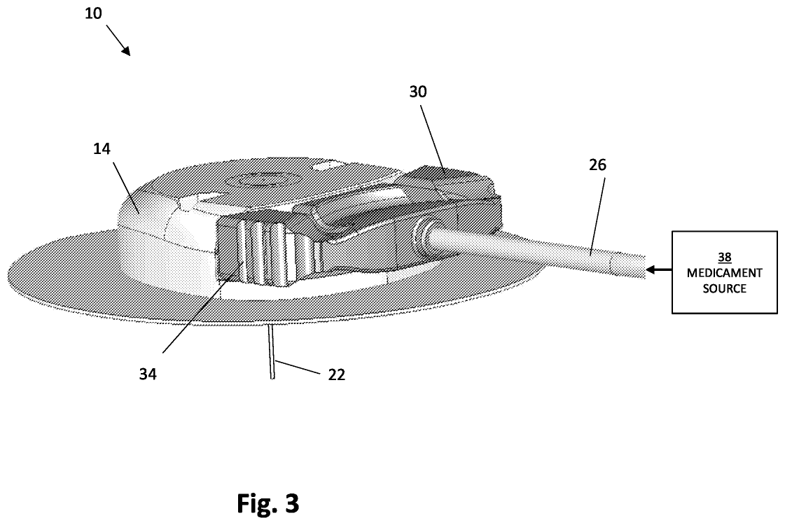

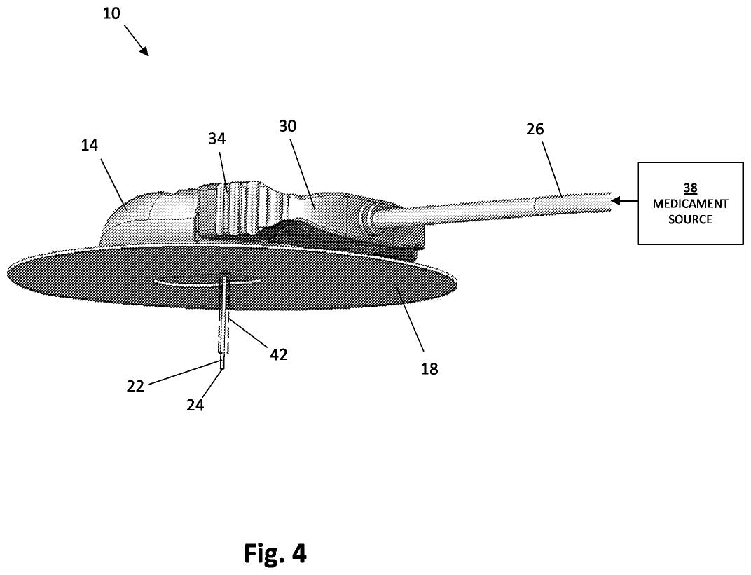

Referring now to FIGS. 3 and 4, the infusion set 10 may include a body 14 having an attachment surface 18 on one side thereof. The attachment surface 18 is configured to contact the infusion site 40 (e.g., skin). In some aspects, the attachment surface 18 may have an adhesive thereon to facilitate attachment of the infusion set 10 to the infusion site 40. The infusion set 10 is configured to receive the medicament from the medicament source 38 and to deliver the medicament to the infusion site 40 via an infusion apparatus.

In some aspects, the infusion apparatus can include a needle 22. The needle 22 can define a needle tip 24 configured to pierce the infusion site 40. The needle 22 can be hollow and define a channel extending therethrough that is configured to receive the medicament therein and to discharge the medicament at the infusion site 40 through the needle tip 24. Actuators and mechanisms may be employed within the body 14 to effect movement of the needle 22 and/or the flexible cannula toward and/or away from the infusion site 40 in a manner understood by those of ordinary skill in the art. Such actuators and mechanisms may be activated by the user from one or more components of the infusion set 10, from the medicament source 38 (e.g., from the infusion pump 50), from another user interface device (not shown), from a combination thereof, and the like. For example, a needle actuator 230 can be actuated to cause movement of the connected infusion apparatus from a retracted position to an injection position.

The infusion set 10 can include a cannula 26 configured to extend between the body 14 and the medicament source 38. The cannula 26 is hollow and defines a channel therethrough configured to receive the medicament from the medicament source 38, to move the medicament therethrough, and to discharge the medicament at, or adjacent to, the infusion site 40 or into the needle 22. In some aspects, the cannula 26 may be configured to be inserted into the infusion site 40, for example, into an aperture of the infusion site 40 created by the needle 22.

The cannula 26 may be attached to the body 14 of the infusion set 10 via one or more coupling mechanisms. As shown in FIGS. 3 and 4, a connector 30 may be disposed on, or adjacent to, the cannula 26 to affix the cannula 26 to the body 14. The connector 30 can attach to the body 14 by any known conventional mechanisms. In some aspects, the infusion set 10 may be designed to allow for decoupling of the connector 30 and the cannula 26 from the body 14.

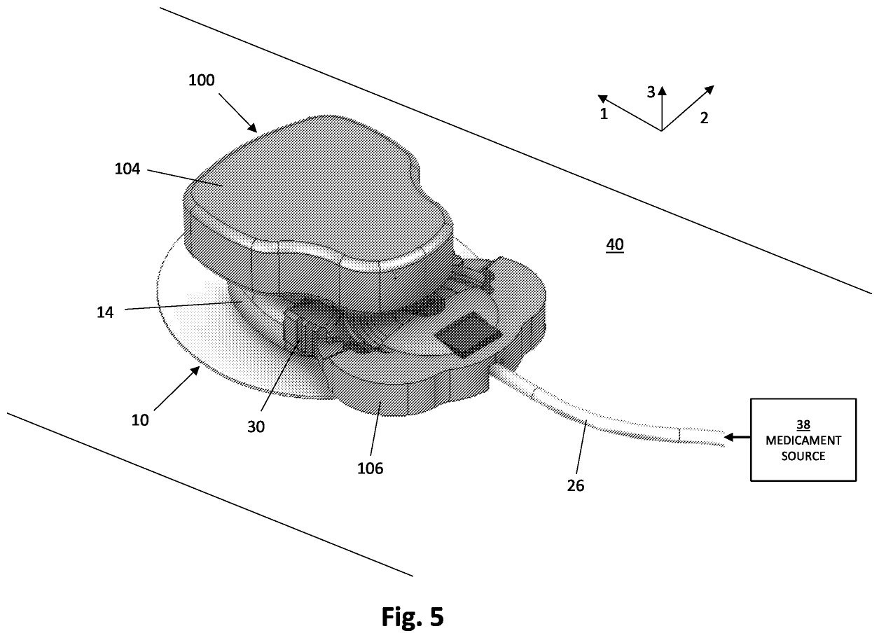

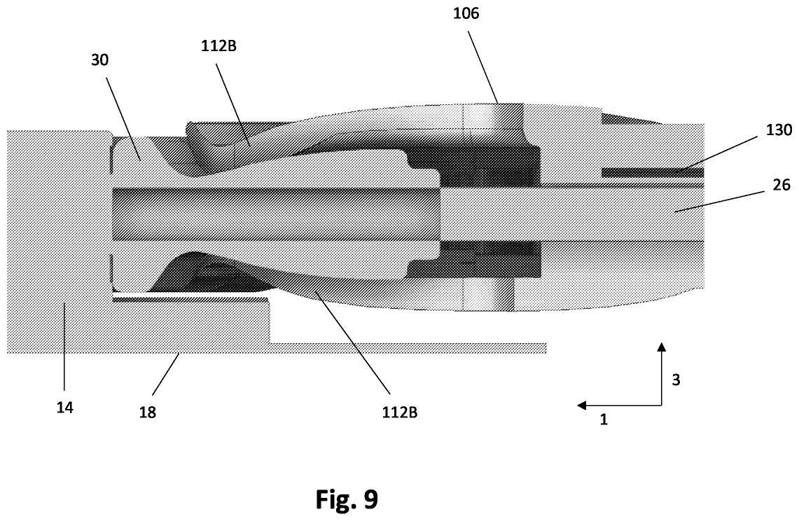

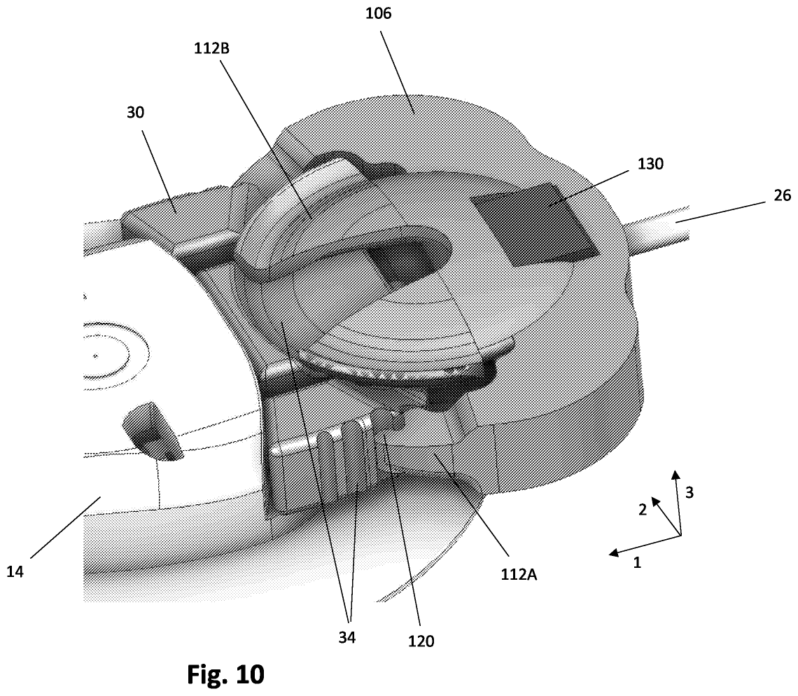

In some aspects, it may be advantageous to monitor one or more infusion parameters of the infusion set 10, the medicament source 38 (e.g., the infusion pump 50), and/or the infusion site 40. Referring to FIGS. 5-12, an infusion set adapter 100 can be operably connected to the infusion set 10. The infusion set adapter 100 can include one or more sensors and controllers, as will be described below, that can provide feedback regarding operation of the infusion set 10 and/or any components connected to the infusion set 10. The feedback can be provided to the user of the infusion set 10 and/or to another destination, such as sending information to a doctor and/or storing information in a database. The infusion set adapter 100 can be configured to prompt an action based on the information collected. The prompted action can include initiating, altering, or terminating infusion of the medicament at the infusion site 40, providing a visual, auditory, and/or haptic signal to the user, sending an electronic notification to the medicament source 38 (e.g., to the infusion pump 50), to a wired or wirelessly connected computing device, and/or to a network database, and/or another predetermined action.

The infusion set adapter 100 can include a housing 104 configured to attach to the infusion set 10. The housing 104 can include a retention member 112 configured to engage with the infusion set 10 to secure the infusion set adapter 100 to the infusion set 10. The retention member 112 can include a clip, hinge, tongue-and-groove, dovetail, resilient protrusion, or another suitable structure to allow for a snap fit, press fit, interference fit, or another engagement between the infusion set 10 and the infusion set adapter 100. In some aspects, the housing 104 may include an adhesive for securing to the body 14 of the infusion set 10. It may be preferable to secure the infusion set adapter 100 to the infusion set 10 in such a way so as to decrease, or entirely prevent, translation and/or rotation of the infusion set adapter 100 relative to at least a portion of the infusion set 10.

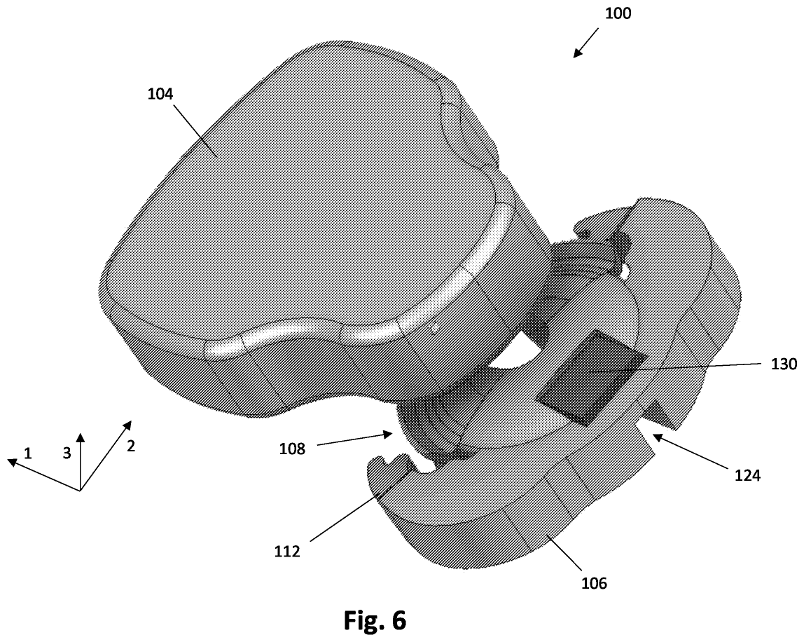

As shown in FIGS. 6-10, the housing 104 may include a plurality of retention members 112 that together can define a receptacle 108 designed to receive the cannula 26 of the infusion set 10. In some aspects, the receptacle 108 may be designed to receive the connector 30 therein. The housing 104 may include retention members 112 disposed at different angles and/or locations relative to the cannula 26 and/or the connector 30. For purposes of this disclosure, the infusion set adapter 100 will be described with reference to a cartesian coordinate system having a first axis extending along a length of the infusion set adapter 100, a second axis extending along a width of the infusion set adapter 100, and a third axis extending along a height of the infusion set adapter 100. Each of the first, second, and third axes is orthogonal to each of the other two axes. Reference will be made to a first direction 1 that is parallel to the first axis, to a second direction 2 parallel to the second axis, and a third direction 3 parallel to the third axis. Each of the first, second, and third directions 1, 2, and 3 can include linear directions in a first linear direction and in a second, opposite linear direction.

As shown in FIGS. 6-8, the housing 104 may include two lateral retention members 112A configured to contact the connector 30 at two opposing sides thereof. The two lateral retention members 112A may be spaced from each other along the second direction 2. Each lateral retention member 112A may be resilient, such that it is configured to be deformed from a first position to a second position upon application of a suitable force, and to move from the second position towards the first position upon removal or reduction of the applied force. When the connector 30 is moved into the receptacle 108 (for example, along the first direction 1), the connector 30 may contact the lateral retention members 112A along a retention section of the connector 30 and may cause the lateral retention members 112A to deflect along the second direction 2 away from the connector 30. As the connector 30 continues to be moved into the receptacle 108, the lateral retention members 112A can slide along the surface of the connector 30 and move towards their respective undeflected states, thus enclosing, at least in part, the connector 30 within the receptacle 108. The lateral retention members 112A may provide a snap fit between the housing 104 of the infusion set adapter 100 and the connector 30 of the infusion set 10. To decouple the connector 30 from the infusion set adapter 100, the connector 30 can be moved out of the receptacle 108 along the first direction 1 away from the receptacle 108. In some aspects, the lateral retention members 112A may be deflected away from the connector 30 prior to moving the connector 30 out of the receptacle 108.

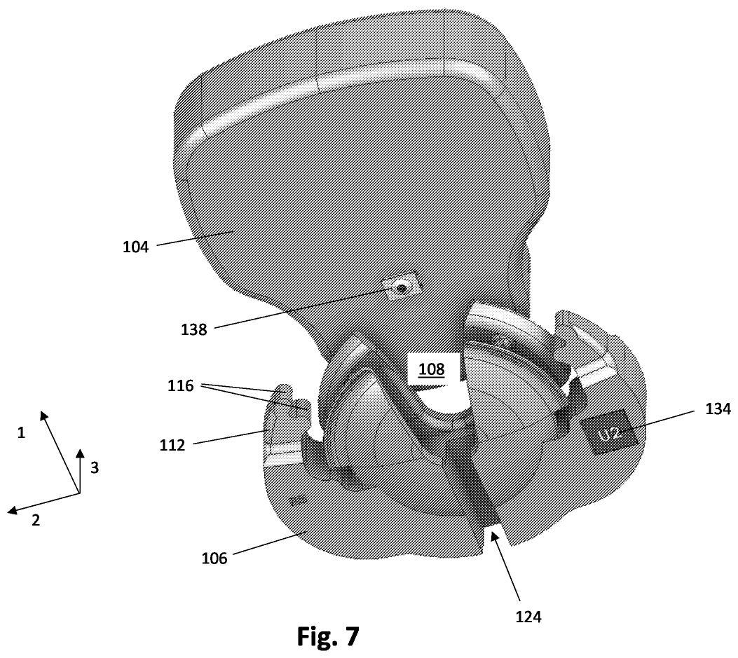

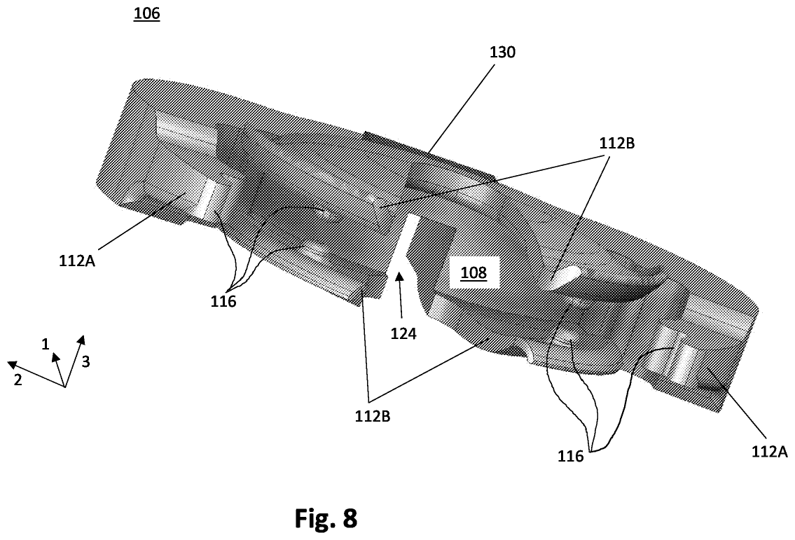

In some aspects, as shown in FIGS. 7 and 8, the lateral retention members 112A may include one or more features 116 thereon configured to engage with corresponding features 34 on the connector 30. The features 116 and 34 may include protrusions, notches, or grooves. The features 116 and 34 may contact each other when the connector 30 is received into the receptacle 108 to further prevent or decrease relative translation and/or rotation of the infusion set adapter 100 relative to the infusion set 10.

The housing 104 may further include at least two opposing vertical retention members 112B spaced from each other along the third direction 3. The vertical retention members 112B can define the receptacle 108. The vertical retention members 112B may function similarly to the lateral retention members 112A described above. When the connector 30 is moved towards the receptacle 108 along the first direction 1, the vertical retention members 112B can be deflected by the connector 30 along the third direction 3 away from the receptacle 108. As the connector 30 is moved into the receptacle 108, the vertical retention members 112B can slide along the connector 30 and move towards their undeflected position along the third direction 3. To decouple the connector 30 from the infusion set adapter 100, the connector 30 can be moved out of the receptacle 108 along the first direction 1 away from the receptacle 108. As the connector 30 is moved out of the receptacle 108, the connector 30 can deflect the vertical retention members 112B away from the receptacle 108 along the third direction 3. In some aspects, the vertical retention members 112B may be deflected away from the connector 30 prior to moving the connector 30 out of the receptacle 108. One or both of the vertical retention members 112B may include one or more features 116 thereon that are configured to engage with the corresponding one or more features 34 on the connector 30.

It may be advantageous to secure the connector 30 in the receptacle 108 between at least the two lateral retention members 112A and between the two vertical retention members 112B. This way, the connector 30 is fixedly secured from translating along, and rotating around, the first, second, and third directions 1, 2, and 3. Because the connector 30 is secured to the body 14 of the infusion set 10, when the connector 30 is prevented from moving relative to the infusion set adapter 100, the body 14 of the infusion set 10 is likewise prevented from movement relative to the infusion set adapter 100.

The housing 104 may include an interface adapter 106 connected thereto for receiving and interfacing with the infusion set 10, for example, with the connector 30. The interface adapter 106 can include the retention members 112 (e.g., the lateral retention members 112A and/or the vertical retention members 112B). Accordingly, the receptacle 108 may be defined by the interface adapter 106, for example, between the interface adapter 106 and the rest of the housing 104. In some aspects, the interface adapter 106 may be removable from the housing 104 and interchangeable with another interface adapter 106. Different interface adapters 106 can be utilized to receive different infusion sets 10, and specifically, different connectors 30. The interface adapter 106 can have any arrangement of the retention members 112 associated with a corresponding shape of the connector 30 or another portion of the infusion set 10.

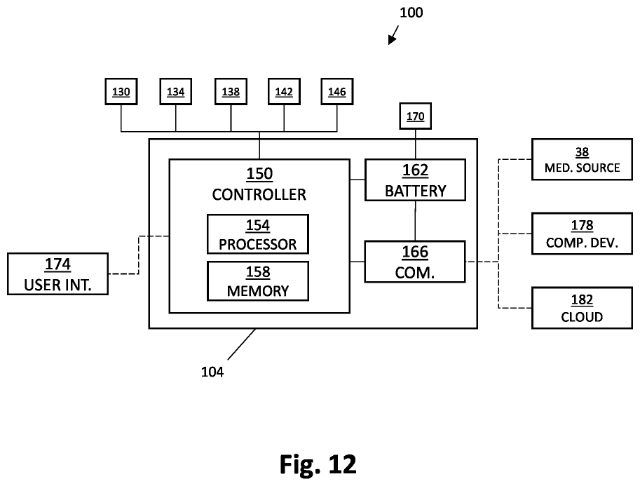

The infusion set adapter 100 further includes a controller 150 configured to communicate with other components of the infusion set adapter 100, with the infusion set 10, with the medicament source 38, and/or with an external computing component. Referring to FIG. 12, the controller 150 can include a printed circuit board (PCB). One or more of the sensors described throughout this application may be mounted to the PCB, or, alternatively, may be spaced from the PCB. The controller 150 may include a processor 154 and a memory 158. The processor 154 may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions that are stored in the memory 158.

The memory 158 may be a single memory device or a plurality of memory devices including but not limited to read-only memory (ROM), random access memory (RAM), volatile memory, non-volatile memory, static random access memory (SRAM), dynamic random access memory (DRAM), flash memory, cache memory, or any other device capable of storing digital information. The memory 158 may also include a mass storage device (not shown) such as a hard drive, optical drive, non-volatile solid state device or any other device capable of storing digital information. The processor 154 may operate under the control of an operating system that resides in the memory 158.

The controller 150 may be operably connected to a user interface 174 to allow a user to interact with the controller 150. The connection between the controller 150 and the user interface 174 may be wired or wireless. The user interface 174 may include one or more input and/or output devices, such as a video monitor, an alphanumeric display, a touch screen, a speaker, an alphanumeric keyboard, a pointing device, a keypad, a pushbutton, a control knob, a microphone, or and any other suitable input or output mechanism capable of providing information to the user and/or receiving information from the user.

The controller 150 may be disposed on the infusion set adapter 100, the infusion set 10, the medicament source 38, or on another component. In some aspects, the controller 150 may include the controller 58 of the infusion pump 50 described above, or, alternatively, the controller 58 of the infusion pump 50 may perform the same tasks as the controller 150. Multiple controllers 150 and/or 58 may be utilized simultaneously.

A communication interface 166 can be operably connected to the controller 150. The communication interface 166 can be configured to send and/or receive signals between the processor 154, the memory 158, and/or any one or more of the sensors described throughout this application. In some aspects, the communication interface 166 can be configured to communicate with additional controllers 150 and/or 58. With continued reference to FIG. 12, the communication interface 166 may be configured to send and/or receive signals between a separate computing device 178, such as a personal computer, a server computer, a workstation, a tablet, a network appliance, a PDA, an e-reader, a cellular phone, a smart watch, or another suitable computing device. In some aspects, the communication interface 166 may be configured to communicate with a network component on a computer network or on a networking cloud 182. In some aspects, the controller 150 may be configured to communicate with the medicament source 38, for example, with the controller 58 on the medicament source 38.

The communication may be via one or more physical wires or may be wireless. The communication may utilize any known wireless transmission protocols, such as Wi-Fi, Bluetooth, NFC, infrared, radio, or other suitable wireless communication method. In some aspects, the wireless connection may be an optical connection, such as a fiber optic connection, wherein a signal can be communicated between components by transmitting a light signal from one component and receiving the light signal by another component.

A power source 162 can be disposed on at least one of the infusion set 10, the infusion set adapter 100, and the medicament source 38. The power source 162 is configured to provide power to the controller 150, the communications interface 166, and/or any connected components, such as one or more of the sensors described further below. The power source 162 can include a battery. The battery can be designed to be disposable or rechargeable. In embodiments with a rechargeable battery, the infusion set adapter 100 may include a charging port 170 (see schematic in FIG. 12) configured to receive charging power from an external power source to charge the battery. The charging port 170 may be configured to receive a charging wire or may be an inductive (i.e., wireless) charging port.

The infusion set adapter 100 can include various mechanisms for monitoring activity related to the infusion set 10. A flow sensor may be used to measure the flow rate of the medicament between the medicament source 38 and the infusion site 40. In some aspects, it may be advantageous to prime the infusion set 10 prior to use. The infusion set 10 is configured to receive the medicament from the medicament source 38, as described above, and to deliver the medicament to the infusion site 40. The medicament can travel through the cannula 26. The infusion set 10 can be primed with the medicament and/or with another suitable priming liquid. The priming process can remove air and/or debris from within the cannula 26 by displacing the air and/or debris with the medicament or another priming liquid. The priming process can also reduce formation and retention of air bubbles within the cannula 26. It may be advantageous to notify the user of the infusion set 10 and/or another individual regarding the priming status of the infusion set 10, for example, whether the infusion set 10 (e.g., the cannula 26) has been primed, whether priming has started, whether priming has finished, and the like.

The infusion set adapter 100 can include a priming sensor 130 configured to be used to detect the priming status of the infusion set 10. The infusion set adapter 100 can also detect the presence of the cannula 26, as well as medicament in the cannula 26 using the priming sensor 130. In some aspects, one or more parameters measured by the priming sensor 130 can be used to quantify how much medicament is present in the cannula 26. The priming sensor 130 can detect and/or measure information that can be used to distinguish between two or more materials in the cannula 26, for example, between the medicament and air.

The priming sensor 130 may be an optical sensor having a light emitter for emitting a light and a light receiver for receiving at least a portion of the emitted light. Light traveling through various media (e.g. air, plastic, water) can have different characteristics, such as different refraction, reflection, wavelengths, and the like. The characteristics of the traveling light through the media can affect how the light is received into the light receiver. The priming sensor 130 can include a known optical sensor that operates by known mechanism. The priming sensor 130 can detect and/or quantify one or more of the characteristics associated with optical measurements of light signals. For example, an optical measurement associated with a light signal passing through only air would correlate to the absence of a cannula adjacent the priming sensor. An optical measurement associated with a light signal passing through plastic and air would correlate to the presence of an unprimed cannula. Finally, an optical measurement associated with a light signal passing through plastic and a liquid would correlate to the presence of a primed cannula.

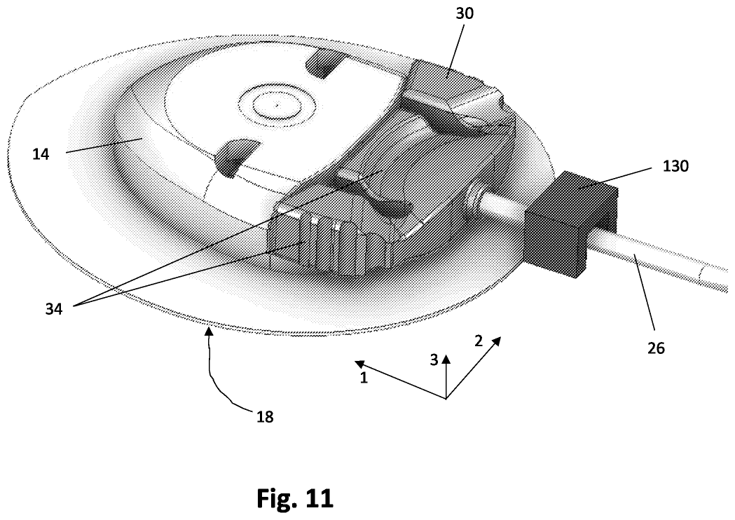

In some aspects, the priming sensor 130 can be disposed on the infusion set adapter 100 such that the cannula 26 is adjacent to the priming sensor 130. It will be appreciated that the cannula 26 can be spaced from the priming sensor 130. As shown in FIGS. 5-11, the priming sensor 130 can be configured to contact the outer surface of the cannula 26 when the infusion set adapter 100 is coupled with the infusion set 10. The housing 104 may include a channel 124 extending therethrough (e.g., on the interface adapter 106) and configured to receive the cannula 26 therein, for example, along the first direction 1. The channel 124 may be defined by the housing 104 (e.g., by the interface adapter 106) and may be dimensioned such that at least a portion of the cannula 26 can be received therein and be disposed adjacent the priming sensor 130. FIG. 11 depicts the priming sensor 130 arranged adjacent to the outer surface of the cannula 26. It will be appreciated that FIG. 11 excludes some components of the infusion set adapter 100 for ease of reference. The housing 104 may include a retention member (not shown) configured to releasably secure the cannula 26 in the channel 124. During use, the priming sensor 130 can be configured to emit a beam of light from one side of the cannula 26 towards the other side of the cannula 26, with the beam of light traveling through the interior of the cannula 26 and through the material therein. When the fluid within the cannula 26 changes, the parameters of the emitted beam of light will also change as the beam of light is received into the light receiver. In some specific applications, the cannula 26 can include air therein prior to receiving the medicament from the medicament source 38. When the medicament is received into the cannula 26 and travels through the cannula 26 so that the emitted beam of light passes through the medicament, the parameters of the detected light change, thus indicating that the medicament is present in the cannula 26 instead of the air. The priming sensor 130 can be used to detect the quantity of medicament in the cannula 26 and compare the detected quantity to the quantity of air present, if any. Continued use of the priming sensor 130 can also indicate presence and quantity of air bubbles within the medicament due to differences in measured light parameters between light passing through air and light passing through the medicament.

In some aspects, the priming sensor 130 can be used to detect if the cannula 26 is properly connected to the medicament source 38. For example, if no medicament is detected, or if a lower amount is detected than expected, such can be an indication of improper connection between the cannula 26 and the medicament source 38 and/or of a leak at the interface of the cannula 26 and the medicament source 38, at the medicament source 38, or at the cannula 26.

In some aspects, the priming sensor 130 can include a flow meter configured to detect and measure flow of a fluid in the cannula 26. The flow meter can be a rotary flow meter having physical components that are moved by the flowing fluid, where each iterative movement of the movable components corresponds to a parameter of fluid flow. The priming sensor 130 can detect and/or measure a characteristic associated with a mechanical flow meter, such as a translation or rotation of a movable component. The flow meter can be a pressure flow meter having a pressure transducer configured to detect the pressure of the fluid flowing through the cannula 26. In some aspects, the priming sensor 130 may include a temperature-based flow sensor configured to measure the temperature of the fluid in the cannula 26. The flow sensor can measure temperature of the fluid at different locations of the cannula 26. The measured temperature can be transmitted, in the form of an electrical signal, to the controller 150, which can analyze the received signal and compare the measurements to determine temperature change. The calculations can be compared to known values corresponding to different types of fluids, such as air, saline, and/or the desired medicament.

In some aspects, the priming sensor 130 may be disposed on the medicament source 38. Alternatively, the priming sensor 130 may be disposed on the cannula 26 itself. Multiple priming sensors 130 may be utilized, for example, along the length of the cannula 26 to determine the priming status of different portions of the cannula 26.

The priming sensor 130 can be configured to contact the cannula 26 (e.g., contact the outer surface of the cannula 26) or may be spaced from the cannula 26. It will be understood that different sensors and sensing technologies can be utilized. In some aspects, as will be described in alternative embodiments below, the priming sensor 130 may be disposed inside a lumen of the cannula 26 and in line with the flow of medicament through the cannula 26. In such aspects, the medicament can flow past the priming sensor 130 toward the infusion site 40. The priming sensor 130 may communicate with the controller 150 disposed on the infusions set adapter 100. The controller 150 will be described further below.

The characteristics detected and/or measured by the priming sensor 130 can be transmitted from the priming sensor 130 to the controller 150 via one or more electronic signals. The processor 154 can receive the signals and store information associated with the received signals in the memory 158. Conversely, the processor 154 may retrieve information from the memory 158, for example, for comparison of received information with stored information.

The controller 150 can process the received or retrieved information based on algorithms, programmed instructions, or other operating instructions provided to the controller 150. The analysis can be used to cause an action to occur based on the received characteristics from the priming sensor 130. The action may include transmitting a signal to another component to cause a task to be performed. The action may also, or alternatively, include presenting a notification indicative of a result of the analysis based on the received information from the priming sensor 130. The controller 150 can transmit the received and/or retrieved information to the one or more connected components listed above.

In some aspects, the priming sensor 130 may be configured to measure a parameter associated with the presence of the cannula 26 adjacent to, or otherwise within operating distance of, the priming sensor 130. The controller 150 can use the measured parameter to determine if the cannula 26 is properly connected to the infusion set 10 and/or to the infusion set adapter 100. A determination that the cannula 26 is not properly connected can be indicative of a missing, disconnected, loose, or damaged cannula 26.

Referring to FIG. 7, the infusion set adapter 100 may include a proximity sensor 134 configured to measure a parameter of relative positioning, placement, and/or orientation of the infusion set adapter 100 and/or the infusion set 10 relative to the infusion site 40. The proximity sensor 134 can measure parameters associated with distances between various components. The measured parameters can be used to determine if the infusion set 10 is properly situated at the infusion site 40 to ensure safe delivery of the medicament to the infusion site 40 and to decrease chances of medicament spills and inaccurate dosing. In scenarios where the infusion set 10 is not fully or properly disposed on the infusion site 40, the user can be notified to correct the placement and/or alignment of the infusion set 10 relative to the infusion site 40 before delivering the medicament. The characteristics detected and/or measured by the proximity sensor 134 can be transmitted from the proximity sensor 134 to the controller 150 via one or more electronic signals. The processor 154 can receive the signals and store information associated with the received signals in the memory 158. Conversely, the processor 154 may retrieve information from the memory 158, for example, for comparison of received information with stored information.

The controller 150 can process the received or retrieved information based on algorithms, programmed instructions, or other operating instructions provided to the controller 150. The analysis can be used to cause an action to occur based on the received characteristics from the proximity sensor 134. The action may include transmitting a signal to another component to cause a task to be performed. The action may also, or alternatively, include presenting a notification indicative of a result of the analysis based on the received information from the proximity sensor 134. The controller 150 can transmit the received and/or retrieved information to the one or more connected components listed above. If the controller 150 determines that the infusion set 10 is improperly located relative to the infusion site 40, the controller 150 may prevent medicament from being delivered from the medicament source 38 to the infusion site 40. The signals received from the proximity sensor 134 can be used to only detect whether the infusion set 10 is properly disposed or not, or, alternatively, to calculate the degree to which the infusion set 10 is improperly positioned (e.g., detect the distance between the infusion set 10 and the infusion site 40). The information received from the proximity sensor 134 can be used to detect if the infusion set 10 and/or infusion set adapter 100 is properly disposed relative to the infusion site 40 prior to initiating movement of the medicament to the infusion site 40. The infusion set adapter 100 may prevent movement of the medicament to the infusion site 40 if the infusion set adapter 100 determines an undesired position of the infusion set adapter 100 and/or the infusion set 10 relative to the infusion site 40. Additionally, or alternatively, the information received from the proximity sensor 134 may be used to detect if the infusion set 10 and/or the infusion set adapter 100 becomes dislodged from, turned, or otherwise moved relative to the infusion site 40 after movement of the medicament has been initiated. In such cases, the infusion set adapter 100 may stop movement of the medicament to the infusion site 40.

The proximity sensor 134 can be configured to contact the infusion site 40 or, alternatively, to be operational when spaced from the infusion site 40. The proximity sensor 134 can utilize any known mechanism for detecting and measuring distance. The proximity sensor 134 may include a contact sensor, which operates by being placed in physical contact with a surface. In such cases, the contact sensor can include at least two components that are movable relative to each other. One of the components can be fixed to the infusion set adapter 100, while the other component can be configured to contact the infusion site 40. Contact with the infusion site 40 can cause the one component to move toward the other component and can form a circuit (e.g., a switch) to indicate the formed contact. Conversely, when the contact sensor is moved away from the infusion site 40, the one component moves away from the other component and disrupts the formed circuit, thus indicating a lack of contact with the infusion site 40. In some aspects, the contact sensor may include a pressure transducer configured to measure a pressure between the contact sensor and the infusion site 40. The measured pressure may be indicative of the degree of contact or orientation between the infusion set 10 and the infusion site 40. For example, the higher the pressure, the greater the contact and connection is between the infusion site 40 and the infusion set 10.

The proximity sensor 134 can include a non-contact sensor, such as an ultrasound sensor, an infrared sensor, an electromagnetic sensor, an optical or photoelectric sensor, an inductive sensor, or a temperature sensor. The proximity sensor 134 can use any of the listed technologies that are known in the art to measure a linear distance between at least two points (e.g., between the infusion set 10 and the infusion site 40). The non-contact sensor can be configured to emit a signal toward the infusion site 40 and to detect and receive a response signal. The response signal can be indicative of the positioning of the infusion set 10 relative to the infusion site 40. For example, the non-contact sensor can emit an ultrasound wave toward the infusion site 40. When the ultrasound wave contacts the infusion site 40, at least a portion of the ultrasound wave rebounds and travels back toward the non-contact sensor. Based on the known values of the speed of ultrasound wave through air, rebound parameters of ultrasound waves from the infusion site 40, amount of ultrasound wave received by the sensor, and the duration of travel of the ultrasound wave, the precise positioning of the infusion set 10 relative to the infusion site 40 can be determined. Similarly, in another aspect, the non-contact sensor can emit a beam of light toward the infusion site 40. When the light contacts the infusion site 40, at least a portion of the light can reflect and travel back to the sensor. Based on known parameters of light emitting and receiving sensors, the precise positioning of the infusion set 10 relative to the infusion site 40 can be determined. In another aspect, the proximity sensor 134 can be configured to detect a temperature immediately adjacent the proximity sensor 134. The detected temperature can be compared to a known temperature value that corresponds to the infusion site 40 being properly distanced from the infusion set 10. The proximity sensor 134 can also determine how far the infusion site 40 is from the infusion set 10 based on the difference between the measured temperature value and the known temperature value. It will be appreciated that the above descriptions are exemplary and that the proximity sensor 134 can utilize any one or more of the listed technologies or another suitable mechanism to determine whether the infusion site 40 is at the desired distance and orientation relative to the infusion set 10. The infusion set adapter 100 may include a plurality of proximity sensors 134 thereon.

In some aspects, it may be advantageous to ensure that the infusion set adapter 100 is properly connected to, and oriented relative to, the infusion set 10. Improper connection can result in inaccurate sensor readings, incorrect signal communication, and/or interference with the desired delivery of the medicament to the infusion site 40. For example, if the infusion set adapter 100 is not fully and correctly engaged with the infusion set 10, the priming sensor 130 may be positioned in such a way that it cannot sufficiently or accurately measure parameters of the flow through the cannula 26. In some cases, improper alignment of the housing 104 (or the interface adapter 106) with the cannula 26 may result in pinching or crimping of the cannula 26, which can result in damage to the cannula 26, medicament leaks, a decreased flow rate of the medicament through the cannula 26, and/or a lower volume of medicament being delivered to the infusion site 40 than desired.

To reduce the likelihood of any of the above problems, the infusion set adapter 100 may include a connection sensor 138 configured to detect a parameter associated with relative positioning of the infusion set adapter 100 and the infusion set 10 and/or relative positioning of the connector 30 and the body 14. The controller 150 can use the detected parameters to determine if the infusion set adapter 100 is properly connected to the infusion set 10 and/or if the connector 30 is properly connected to the body 14. If the controller 150 detects that the connection is inadequate, the controller 150 may send a notification to the user to instruct the user to correct the connection. If connection is determined to be improper, the controller 150 can preclude the infusion set 10 from receiving the medicament from the medicament source 38 until parameters detected and/or measured by the connection sensor 138 result in the controller 150 determining that the connection between the infusion set 10 and the infusion set adapter 100 and/or the connector 30 and the body 14 is as desired. The characteristics detected and/or measured by the connection sensor 138 can be transmitted from the connection sensor 138 to the controller 150 via one or more electronic signals. The processor 154 can receive the signals and store information associated with the received signals in the memory 158. Conversely, the processor 154 may retrieve information from the memory 158, for example, for comparison of received information with stored information. The controller 150 can process the received or retrieved information based on algorithms, programmed instructions, or other operating instructions provided to the controller 150. The controller can transmit a signal to another component to cause a task to be performed based on the analysis of the received information from the connection sensor 138, as described above. The controller 150 can transmit the received and/or retrieved information to the one or more connected components listed throughout this application.

Referring to FIG. 7, the connection sensor 138 may be disposed on the infusion set adapter 100, and specifically on a surface that is intended to be adjacent to the infusion set 10 when the infusion set adapter 100 is connected thereto. The connection sensor 138 may be similar to, or substantially the same as, the proximity sensor 134 described above. That is, the connection sensor 138 may include any of the contact sensors technologies or non-contact sensor technologies described above. The controller 150 may be configured to utilize the received characteristic measurements from the connection sensor 138 to detect between two states of the infusion set 10 and/or the infusion set adapter 100: connected and not connected. In some aspects, the controller 150 may be configured to detect a degree to which the infusion set adapter 100 is connected to or disconnected from the infusion set 10 and/or the connector 30 is connected to the body 14. For example, if the controller 150 determines, based on the characteristics received from the connection sensor 138, that the infusion set adapter 100 is not properly connected to the infusion set 10, the controller 150 may be able to quantify how far away the infusion set adapter 100 is away from the infusion set 10 and/or identify a reason for the improper connection (e.g., components are spaced too far apart, components are oriented incorrectly relative to each other, there is presence of debris or another component that interferes with proper connection, and the like).

The connection sensor 138 may be configured to detect a parameter associated with the connection of the interface adapter 106 with the rest of the housing 104. The controller 150 can receive the detected parameter and use the detected parameter to determine if the interface adapter 106 is properly connected to the housing 104. In some aspects, the controller 150 may be configured to utilize the received parameter to determine if the correct interface adapter 106 is connected to the housing 104.

In some aspects, the connection sensor 138 may be configured to detect a characteristic associated with a connection between the medicament source 38 and the infusion set adapter 100. The controller 150 may receive the measured characteristics from the connection sensor 138 and perform a calculation to determine if the medicament source 38 is properly connected to the infusion set 10 and/or the infusion set adapter 100. The controller 150 can perform an action based on the measured characteristic of the connection sensor 138, for example, to notify the user if/when the connection between the medicament source 38 and the infusion set 10 (e.g., between the infusion pump 50 and the cannula 26) is formed, is terminated, or is altered. This can prevent spills of medicament due to poor connection and can avoid inaccurate dosing.

In some aspects, it may be advantageous to detect and monitor a part of the infusion process that initially introduces the infusion set 10 and/or the medicament to the infusion site 40. This process can include utilizing the needle 22 (described above) to pierce the infusion site 40 (e.g., skin) to form an aperture therein to receive the medicament. The needle 22 can then be removed, and the medicament can be introduced to the infusion site 40 through the formed aperture. The medicament can be introduced by inserting a portion of the cannula 26 into the aperture. In some aspects, as described above, the needle 22 may be hollow and configured to receive the medicament therein from the cannula 26 and to deliver the medicament therethrough to the infusion site 40. In such aspects, the needle 22 may be kept at the infusion site 40 after forming the aperture instead of being removed.

It may be preferable to detect the presence of the needle 22 and/or the cannula 26 at the infusion site 40 to ensure that the aperture is formed as desired and that the medicament can be delivered thereto. If the needle 22 is not moved to its intended distance toward and through the infusion site 40, the desired aperture size and depth may not be formed, which can result in delivery of an insufficient amount of the medicament or to delivery of the medicament to an incorrect location. If the needle 22 and/or the cannula 26 is not retained within the formed aperture at the infusion site 40 at the desired location and/or orientation relative to the infusion set 10, the medicament can be prevented from reaching the intended target at the infusion site 40 and can result in less medicament being delivered than needed and/or spills of the medicament.

The infusion set adapter 100 may include a needle sensor 142 (see schematic of FIG. 12) configured to measure a parameter of the position of the needle 22, and specifically of the needle tip 24, relative to one or more components described herein (e.g., the infusion set 10, the infusion set adapter 100, or the infusion site 40). The controller 150 can utilize the measured parameter to detect movement of the needle 22 relative to the infusion set adapter 100, the infusion set 10, and/or the infusion site 40. The needle 22 can be movable between a sheathed configuration and an unsheathed configuration. In the sheathed configuration, the needle 22 can be retracted and is positioned away from the infusion site 40, for example, within the body 14 of the infusion set 10. In the unsheathed configuration, the needle 22 can be extended, and at least a portion of the needle 22 may be outside of the body 14 and configured to contact and pierce the infusion site 40. The controller 150 may be configured to utilize parameters provided by the needle sensor 142 to detect whether the needle 22 is in the sheathed or unsheathed configuration. The controller 150 can be configured to detect when the needle 22 is moved between the sheathed and the unsheathed configurations. The controller 150 can calculate a distance that the needle 22 is moved when transitioning from the sheathed configuration to the unsheathed configuration or vice versa. The characteristics detected and/or measured by the needle sensor 142 can be transmitted from the needle sensor 142 to the controller 150 via one or more electronic signals. The processor 154 can receive the signals and store information associated with the received signals in the memory 158. Conversely, the processor 154 may retrieve information from the memory 158, for example, for comparison of received information with stored information. The controller 150 can process the received or retrieved information based on algorithms, programmed instructions, or other operating instructions provided to the controller 150. The analysis can be used to cause an action to occur based on the received characteristics from the needle sensor 142. The action may include transmitting a signal to another component to cause a task to be performed. The action may also, or alternatively, include presenting a notification indicative of a result of the analysis based on the received information from the needle sensor 142. The controller 150 can transmit the received and/or retrieved information to the one or more connected components listed above.

The needle sensor 142 can include an optical sensor, for example any optical sensor described throughout this application. The optical sensor can detect presence of the needle 22 (or a particular feature of the needle 22) to identify a relative location of the needle 22 based on known or prerecorded reference values. For example, the needle 22 may include an identification feature (not shown) thereon that can be detected and identified based on the characteristics measured by the needle sensor 142. Detection of the identification feature can be indicative of the needle 22 being disposed at a first position relative to the infusion set 10, the infusion set adapter 100, and/or the infusion site 40. The needle 22 may include a plurality of identification features, with each identification feature corresponding to a different position of the needle 22.

In another aspect, the needle sensor 142 may include a mechanical sensor configured to detect a parameter associated with the relative position of the needle 22 and measure a distance that the needle 22 is moved. The mechanical sensor may include an odometer, waywiser, or a similar mechanism configured to detect movement of the needle 22 and to quantify said movement. It should be appreciated that any suitable distance measuring mechanism can be utilized.

In some embodiments where the medicament may be delivered to the infusion site 40 via direct contact with the cannula 26, or via a second cannula (not shown) in fluid communication with the cannula 26, (instead of through the needle 22) the needle sensor 142 may be configured to measure a parameter associated with the relative positioning of the cannula 26 (or the second cannula) relative to the infusion set 10, the infusion set adapter 100, and/or the infusion site 40.

In some aspects, the infusion set 10 and/or the infusion set adapter 100 may include a needle guard 42 (shown in phantom in FIG. 4). The needle guard 42 can include a hollow tubular structure that is configured to slidably receive the needle 22 therein. The needle 22 may be configured to move relative to the needle guard 42, and/or the needle guard 42 may be configured to move relative to the needle 22. The needle guard 42 can receive the needle 22 therein such that the needle tip 24 is within the needle guard 42. When the needle tip 24 is within the needle guard 42, the needle 22 may be in the sheathed configuration. The needle 22 can be moved out of the needle guard 42 (or the needle guard 42 can be moved relative to the needle 22) such that the needle tip 24 is outside of the tubular structure of the needle guard 42. When the needle tip 24 is not within the needle guard 42, the needle 22 can be in the unsheathed configuration.

The needle guard 42 can be rigidly affixed to the body 14 of the infusion set 10, for example adjacent to the attachment surface 18. In some aspects, the needle guard 42 may be movable relative to the body 14, for example, slidably movable towards the body 14, such that at least a portion of the needle guard 42 is movable within the body 14.

The infusion set adapter 100 may include a needle guard sensor 146 configured to detect and/or measure a characteristic associated with the relative position of the needle 22. The measured characteristic can be transmitted to the controller 150, which can calculate the position of the needle guard 42 relative to the needle 22 (for example, specifically the needle tip 24), the infusion site 40, and/or the infusion set body 14 (see schematic in FIG. 12). The needle guard sensor 146 may include any of the sensor mechanisms and technologies described throughout this application with respect to any of the other listed sensors. The characteristics detected and/or measured by the needle guard sensor 146 can be transmitted from the needle guard sensor 146 to the controller 150 via one or more electronic signals. The processor 154 can receive the signals and store information associated with the received signals in the memory 158. Conversely, the processor 154 may retrieve information from the memory 158, for example, for comparison of received information with stored information. The controller 150 can process the received or retrieved information based on algorithms, programmed instructions, or other operating instructions provided to the controller 150. The analysis can be used to cause an action to occur based on the received characteristics from the needle guard sensor 146. The action may include transmitting a signal to another component to cause a task to be performed. The action may also, or alternatively, include presenting a notification indicative of a result of the analysis based on the received information from the needle guard sensor 146. The controller 150 can transmit the received and/or retrieved information to the one or more connected components listed above.

In some aspects, the needle 22 may be configured to be retained within the body 14 such that the needle tip 24 does not protrude past the attachment surface 18 when the needle 22 is in the sheathed configuration. In such aspects, the body 14 may serve a similar role as the needle guard 42, and the infusion set 10 may be devoid of a separate needle guard 42 such as described above.

The infusion set adapter 100 and/or the infusion set 10 can include any other suitable sensors for detecting and/or measuring parameters of medicament infusion, such as humidity, leaks, and the like. For example, a liquid sensor (not shown) can include one or more electrodes configured to form a circuit when contacted by a liquid, thus indicating a leak. The infusion set adapter 100 may include a flow sensor (not shown) adjacent the infusion site 40 to detect a parameter associated with the amount of medicament flowing out of the infusion set 10 to the infusion site 40. The infusion set adapter 100 may include a flow sensor (not shown) at the medicament source 38 configured to detect a parameter associated with the amount of the medicament being moved from the medicament source 38 to the cannula 26.

In some aspects, the infusion set adapter 100 may include an air bubble sensor (not shown) configured to detect and/or quantify the presence of air bubbles in the cannula 26, the needle 22, or another component of the infusion set 10, infusion set adapter 100, and/or medicament source 38. It may be advantageous to identify and remove air bubbles to prevent introduction of air into the infusion site 40 and to ensure that an accurate amount of medicament is delivered. The additional sensors can be configured to transmit electronic signals to the controller 150, and the controller 150 can analyze and process the signals. The controller 150 can cause an action to occur in response to the analysis of the received information from the sensors. It will be appreciated that the characteristic received by the controller can be indicative of any parameter that the particular sensor is configured to measure. For example, if the sensor is configured to measure movement of a mechanical portion or application of a pressure to the sensor, then the characteristic may be indicative of presence of a contact between two components. If the sensor is configured to measure temperature, then the characteristic received by the controller 150 may be indicative of the temperature of the particular location being measured. In some aspects, while air bubbles may not be actively removed from the medicament, it may be preferable to account for the amount of air within the medicament. This can allow the controller 150 to determine how much of the total volume of flow moving from the medicament source 38 to the infusion site includes the medicament and how much includes air. Such calculations can be used to exclude the calculated volume of air from the volume of medicament being moved to the infusion site 40 to ensure that an accurate and precise amount of medicament is delivered to the infusion site 40.

In some aspects, one or more wires can be used for communication between components. A wire (not shown) can be operably connected to the controller 150 at one end thereof and to another component of the infusion set 10, infusion set adapter 100, or medicament source 38 at another end thereof. A plurality of wires can be used to communicate between multiple components. Wires can be used instead of, or in addition to, a wireless means of communication.

In some aspects, the wire can extend between the infusion set adapter 100 and the medicament source 38 (e.g., the infusion pump 50). The wire can be received into a separate wire conduit (not shown) to protect the wire from damage, moisture, interference, and the like. The separate wire conduit can be spaced from the cannula 26.

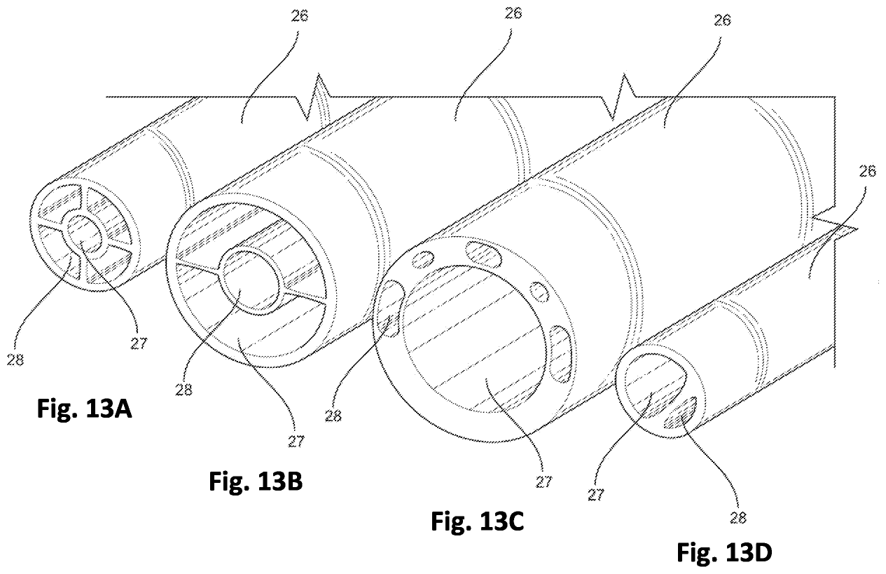

In some aspects, the wire can be received into or onto the cannula 26. Referring to FIGS. 13A-13D, the cannula 26 can include a first lumen 27 extending along the length of the cannula 26. The first lumen 27 can be the medicament conduit. The first lumen 27 is configured to receive the medicament therein from the medicament source 38 and to discharge the medicament therefrom at the infusion set 10 or at the infusion site 40. The cannula 26 may include a second lumen 28 extending therethrough. The second lumen 28 can be the wire conduit. The second lumen 28 may extend along the entirety of the length of the cannula 26, or, alternatively, may extend along a portion of the length of the cannula 26. The second lumen 28 is configured to receive one or more wires therein. The wires can be connected to the controller 150 and/or to any one or more of the sensors described above at one end thereof, and to the medicament source 38 at the other thereof. It should be understood that the second lumen 28 should be separated from the first lumen 27 to so as to protect the wire from the medicament and to ensure cleanliness and sterility of the medicament. The first lumen 27 may be positioned radially within the second lumen 28 (see FIG. 13A). Alternatively, the second lumen 28 may be positioned radially within the first lumen 27 (see FIG. 13B). Alternatively, the second lumen 28 may be positioned adjacent to, but not within, the first lumen 27 (see FIG. 13D). In some aspects, the cannula 26 may include a plurality of second lumens 28 and/or first lumens 27. The plurality of second lumens 28 may be disposed circumferentially around the first lumen 27 (see FIG. 13C). It will be appreciated that other suitable arrangements of singular or plurality of first and second lumens 27 and 28 are envisioned.

The controller 150 may be configured to cause one or more actions to occur in response to a signal received from any one or more of the sensors or other components described above. In some aspects, the controller 150 can communicate one or more signals to connected components to cause one or more actions with the infusion set adapter 100, infusion set 10, and/or medicament source 38. For example, if the priming sensor 130 detects that the cannula 26 is not properly primed as desired, the controller 150 may communicate a signal to the medicament source 38 (e.g., to the infusion pump 50) to cause delivery of medicament into the cannula 26 until the priming sensor 130 detects that the desired priming has been achieved. This can allow for the desired duration of medicament infusion and a desired amount of medicament delivered. The controller 150 may operate parameters of the medicament delivery, for example, by operating the infusion pump 50. The controller 150 can initiate pumping of the medicament from the medicament source 38 by the infusion pump 50, can initiate termination of pumping of the medicament, and/or can change the pumping rate of the medicament.

The controller 150 may transmit a signal to the infusion set 10 to selectively permit or preclude introduction of the needle 22 into the infusion site 40. If the priming sensor 130 detects that the cannula 26 is not sufficiently primed, the controller 150 may be configured to prevent the needle 22 from being moved towards the infusion site 40 to pierce the infusion site 40. This can prevent inadvertent introduction of air and/or debris into the infusion site 40 from the unprimed cannula 26. Conversely, if the priming sensor 130 detects that the cannula 26 is sufficiently primed, the controller 150 can permit the needle 22 to be moved towards the infusion site 40 and to pierce the infusion site 40, thus placing the infusion site 40 in liquid communication with the primed cannula 26 with the medicament therein. In some aspects, the controller 150 may be configured to actively cause the needle 22 to be moved towards the infusion site 40.

If the priming sensor 130 detects that the cannula 26 is not primed, the controller 150 may be configured to calculate the amount of medicament or other priming fluid that needs to be introduced into the cannula 26 to achieve the desired priming state, and the controller 150 can instruct the infusion pump 50 to pump the needed amount of medicament to the cannula 26 until the priming sensor 130 determines that the desired priming is achieved. The controller 150 may be configured to transmit a signal to the infusion set adapter 100, the medicament source 38, the computing device 178, and/or the network cloud 182 to trigger an alert to notify the user of the status of the priming sensor 130 and of the related actions described above. The alert can include auditory, visual, and/or tactile notifications for the user and/or for another individual.

The controller 150 may operate components based on signals received from the proximity sensor 134. For example, if the proximity sensor 134 detects that the infusion set 10 is not adequately contacting the infusion site 40, the controller 150 may preclude movement of the needle 22 towards, and into, the infusion site 40. This may prevent inadvertent movement of the needle 22 from the sheathed configuration to the unsheathed configuration before the infusion set 10 is properly attached to the infusion site 40. Premature unsheathing of the needle 22 can cause injury to the user.

The controller 150 may also prevent movement of the medicament towards the infusion site 40 from the medicament source 38. If the infusion set 10 is not in the desired position relative to the infusion site 40, medicament that is moved therethrough can be inadvertently discharged out of the infusion set 10 at an undesired location and not be delivered to the infusion site 40. This can result in inaccurate dosing of the user, as well as undesired spills. The controller 150 may be configured to transmit a signal to the infusion set adapter 100, the medicament source 38, the computing device 178, and/or the network cloud 182 to trigger an alert to notify the user of the status of the proximity sensor 134 and of the related actions described above. The alert can include auditory, visual, and/or tactile notifications for the user and/or for another individual.

The controller 150 may operate components based on signals received from the connection sensor 138. The infusion set adapter 100 should be properly connected to the infusion set 10 as described previously. Improper connection or alignment can result in inaccurate readings from the various sensors (e.g., the priming sensor 130 or the proximity sensor 134), kinking or other obstruction of the cannula 26, and/or discomfort for the user due to undesired contact with the misaligned components. If the connection sensor 138 detects that the infusion set adapter 100 is not sufficiently connected to, or properly aligned with, the infusion set 10, the controller 150 may preclude delivery of the medicament to the infusion site 40 until the relative position between the infusion set adapter 100 and the infusion set 10 is corrected. In some aspects, the controller 150 may be configured to determine the reason why the infusion set adapter 100 is not properly connected with the infusion set 10 (e.g., spaced too far away, oriented at an incorrect angle, or prevented from connecting by an external component). The controller 150 may transmit a signal to the infusion set adapter 100, the medicament source 38, the computing device 178, and/or the network cloud 182 to trigger an alert to notify the user of the status of the connection sensor 138 and of a detected reason for the improper connection. The alert can include auditory, visual, and/or tactile notifications for the user and/or for another individual.

The controller 150 may operate components based on signals received from the needle sensor 142. If the needle sensor 142 detects that the needle 22 is in its unsheathed position, the controller 150 can transmit a signal to the medicament source 38 to cause the medicament to be moved therefrom into and through the cannula 26 toward the infusion site 40. For example, the controller 150 can instruct the infusion pump 50 to pump the medicament through the cannula 26. If the needle sensor 142 detects that the needle 22 is in its sheathed configuration, the controller 150 may send a signal precluding movement of the medicament towards the infusion site 40 to avoid premature delivery of the medicament. This can reduce spills. This can also ensure that medicament is not delivered to a location other than the intended infusion site 40, and thus allows for more accurate measurement of the medicament dosage at the infusion site 40.

In some aspects, the needle sensor 142 may detect that, although the needle 22 is in its unsheathed configuration, the needle tip 24 is not at the desired position. In such cases, the controller 150 can transmit a signal to the infusion set 10 to cause the needle 22 to be moved to the desired position. This can be advantageous to ensure that the needle 22 is moved far enough into the infusion site 40 to effectively pierce the infusion site 40 and to place the needle tip 24 at the location within the infusion site 40 where the medicament is desired.

The controller 150 may be configured to transmit a signal to the infusion set adapter 100, the medicament source 38, the computing device 178, and/or the network cloud 182 to trigger an alert to notify the user of the status of the needle sensor 142 and of the related actions described above. The alert can include auditory, visual, and/or tactile notifications for the user and/or for another individual.

The controller 150 may operate components based on signals received from the needle guard sensor 146. The needle guard sensor 146 can detect if the needle 22 is in its sheathed or unsheathed configuration, as well as the position of the needle tip 24 relative to the infusion set 10 and the needle guard 42. If the needle guard sensor 146 detects that the needle tip 24 is within the needle guard 42 (i.e., the needle tip 24 is not exposed), the controller 150 can notify the user that the needle 22 is safely positioned, and risk of injury is decreased. If the needle guard sensor 146 detects that the needle tip 24 is outside of the needle guard 42 (i.e., the needle tip 24 is exposed), the controller 150 can notify the user of the risk of injury from the exposed needle tip 24. The controller 150 can transmit a signal to the infusion set adapter 100, the medicament source 38, the computing device 178, and/or the network cloud 182 to trigger such alerts or notifications. The alerts can include auditory, visual, and/or tactile notifications for the user and/or for another individual.

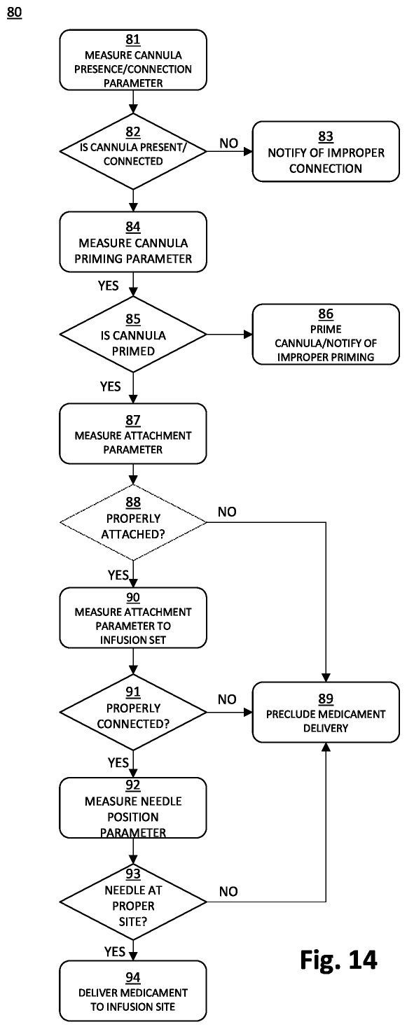

An operating process 80 is depicted in FIG. 14, that shows an exemplary logic schematic of operating the infusion set adapter 100. It should be appreciated that the depicted steps are exemplary and can be performed in any order, for any number of iterations, and may include additional steps not depicted in FIG. 14.

In step 81, the priming sensor 130 may detect a parameter associated with the presence of the cannula 26. In step 82, the controller 150 can use the parameter from step 81 to determine whether the cannula 26 is present and/or properly connected to the infusion set 10 and/or is properly disposed relative to the infusion set adapter 100, as described previously. The controller 150 is configured to receive information from the priming sensor 130 to determine whether the cannula is present and/or the connection with the cannula is acceptable. If the controller 150 determines that the cannula 26 is not present or that the cannula 26 is not properly connected, the process can proceed to step 83, where the controller 150 can notify the user of the missing cannula 26 or of the improper connection of the cannula. The controller can additionally or alternatively cause a different action in step 83 associated with the determination from step 82.

If the controller 150 determines that the state of connection between the cannula 26 and the infusion set 10 and/or the infusion set adapter 100 is acceptable, the process can proceed to step 84, in which the priming state of the infusion set 10 is detected. Specifically, the priming state of the cannula 26 may be detected by the priming sensor 130 as described above. The priming sensor 130 can detect and/or measure a parameter associated with the priming state of the cannula 26. After the parameter is detected and/or measured, the process 80 can proceed to step 85, where the controller 150 can use the detected and/or measured parameter from step 84 to determine whether the cannula 26 is properly primed. In step 85, the controller 150 is configured to receive information from the priming sensor 130. If the controller 150 determines that the cannula 26 is not primed, the process 80 can proceed to step 86, where the controller 150 can instruct the medicament source 38 to prime the cannula 26 (e.g., by operating the infusion pump 50 to pump the medicament into the cannula 26). The controller 150 may additionally, or alternatively, transmit a notification to the user and/or to another destination or component indicating the priming status of the cannula 26. If, in step 85, the controller 150 determines that the cannula 26 is properly primed, the process can proceed to step 87.

In step 87, the proximity sensor 134 can detect a parameter associated with the attachment of the infusion set 10 with the infusion site 40. After the parameter is detected and/or measured, the process can proceed to step 88, where the controller 150 can use the parameter from step 87 determine the state of attachment of the infusion set 10 with the infusion site 40. The controller 150 is configured to receive information from the proximity sensor 134. In step 88, the controller 150 can determine whether infusion set 10 is properly attached to, and/or properly oriented relative to, the infusion site 40. The particular desired attachment can include a threshold percentage of the attachment surface 18 being in contact with the infusion site 40. In some aspects, the desired attachment can include an acceptable distance range measured between the infusion site 40 and the proximity sensor 134. The desired attachment and related values can be predetermined and programmed into the controller 150 (e.g., into the memory 158).

If the controller 150 determines that the infusion set 10 is not attached to the infusion site 40 as desired, the process 80 can proceed to step 89. In step 89, the controller 150 can preclude movement of the medicament to the infusion site 40. The controller 150 can transmit a signal to any one or more of the infusion set 10, infusion set adapter 100, medicament source 38, connected computing device 178, cloud network 182, or another wired or wirelessly connected component. For example, the controller 150 can transmit a signal to the medicament source 38 to preclude the infusion pump 50 from being actuated to pump the medicament through the cannula 26. The signal sent from the controller 150 can be desired to trump a contradictory signal. For example, if the user attempts to actuate pumping of the medicament by the infusion pump 50, the signal sent from the controller 150 in step 89 can override the user input to prevent operation of the infusion pump 50. Such operational control can be advantageous in situations where the user may not be aware of the poor attachment between the infusion set 10 and the infusion site 40 so as to prevent inadvertent delivery of the medicament to the incorrect location.

If the controller 150 determines that the attachment between the infusion set 10 and the infusion site 40 is adequate, the process 80 may proceed to step 90. In step 90, the connection sensor 138 can detect a parameter associated with the connection between the infusion set adapter 100 and the infusion set 10 as described previously. After the parameter is detected, the process can proceed to step 91, where the controller 150 can determine if the connection between the infusion set adapter 100 and the infusion set 10 is correct using the parameter from step 90. The controller 150 can include known threshold values and/or ranges of values pertaining to what should be construed as proper connection and what is an improper connection. The proper connection can include an acceptable distance range measured between the infusion set 10 and the infusion set adapter 100 via the connection sensor 138. Such ranges and values can be predetermined and programmed into the controller 150 (e.g., into the memory 158).

If the controller 150 determines that the connection between the infusion set 10 and the infusion set adapter 100 is not sufficient, the process 80 can proceed to step 89, in which the medicament is precluded from being delivered to the infusion site 40, as described above.

If the controller 150 determines that the connection between the infusion set 10 and the infusion set adapter 100 is sufficient, the process 80 can proceed to step 92. In step 92, the needle sensor 142 can detect a parameter associated with the presence of the needle 22. The process 80 can then proceed to step 93, where the controller 150 can use the parameter from step 92 to detect/determine the presence and relative position of the needle 22 as described previously. The needle sensor 142 can specifically detect the location of the needle tip 24 relative to the infusion set 10, the infusion site 40, and/or the needle guard 42.

In step 93, the controller 150 can determine if the needle 22 is at its desired location. The controller 150 can include known threshold values and/or ranges of values pertaining to what should be construed as a proper location and what is an improper location of the needle 22 or the needle tip 24. The proper location can include an acceptable distance range measured between the infusion set 10 and the infusion site 40. For example, the distance range can be measured between the attachment surface 18 of the infusion set 10 and the needle tip 24 of the needle 22. Such ranges and values can be predetermined and programmed into the controller 150 (e.g., into the memory 158).

If the controller 150 determines that the needle 22 is not in the desired location, the process 80 may proceed to step 89, in which the medicament is precluded from being moved to the infusion site 40, as described above.

If the controller 150 determines that the needle 22 is in the desired location, the process 80 may proceed to step 94.

In step 94, if all other parameters are met, the medicament can be delivered to the infusion site 40. The controller 150 can transmit a signal to any one or more of the infusion set 10, infusion set adapter 100, medicament source 38, connected computing device 178, cloud network 182, or another wired or wirelessly connected component. For example, the controller 150 can transmit a signal to the medicament source 38 to actuate the infusion pump 50 to pump the medicament through the cannula 26 toward the infusion site 40. The controller 150 can transmit one or more signals to actively initiate or continue movement of the medicament toward the infusion site 40. Alternatively, the controller 150 can transmit a signal to remove any preclusions that were previously conveyed, such as those in step 88 described above. When the preclusions have been removed, the user can then actuate delivery of the medicament from the medicament source 38, for example, by actuating the drive system 56 on the infusion pump 50 to cause the medicament stored in the reservoir 52 to be pumped, via the drive system 56, into and through the cannula 26.

It should be understood that the process 80 can include additional steps, and one or more of the steps described above can be performed in a different order. Furthermore, the steps described above can be ordered in such a way that one or more of the described steps determine whether and when the process 80 continues to any other step.

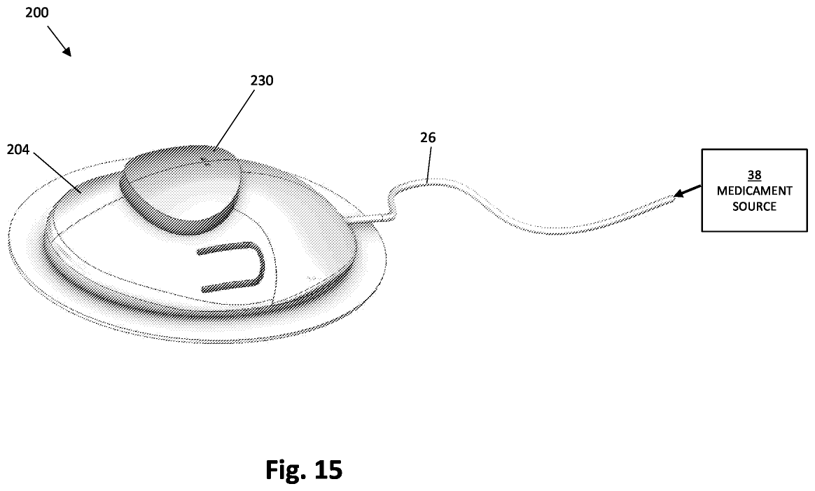

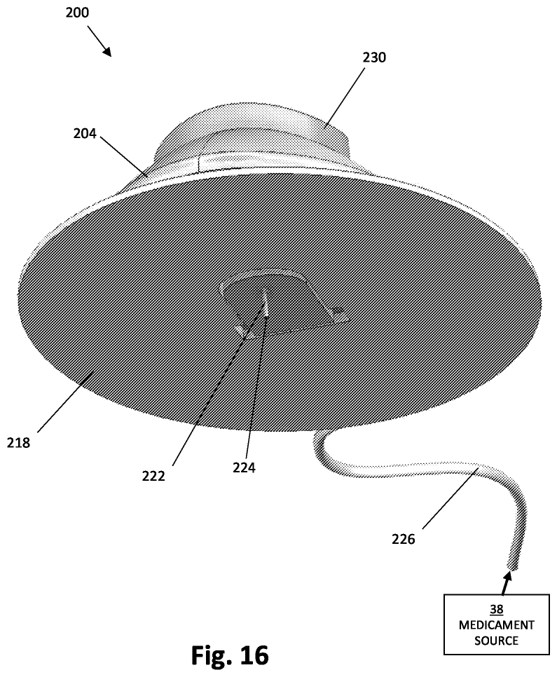

In some embodiments, the infusion set adapter 100 (or the components thereof) can be integral with the infusion set 10. Referring to FIGS. 15-18, an infusion set 200 is depicted. Elements and components of the infusion set 200 can be similar to, or substantially the same as, elements and components of the infusion set 10 and/or the infusion set adapter 100 described previously. Descriptions of the features with respect to the infusion set 10 and/or the infusion set adapter 100 can apply to similar features of the infusion set 200.

The infusion set 200 can include some or all of the components of the infusion set 10 and the infusion set adapter 100 described above. While the infusion set adapter 100 can be designed to be separated from the infusion set 10 and to operate with one or more different types of infusion sets 10, the infusion set 200 incorporates features of both, the infusion set 10 and the infusion set adapter 100, into a single integrated unit. Such a design may be advantageous to simplify attachment and use. For example, the infusion set 200 can be the only device applied to the infusion site 40 by the user, thus reducing the number of separate devices that the user must handle. This can reduce user error, complexity of use, and user anxiety. Additionally, because the infusion set 200 includes components of both the infusion set 10 and the infusion set adapter 100, a separate infusion set adapter 100 may not be needed. This can reduce the risk of improperly attaching the infusion set adapter 100 to the infusion set 10 as described above.

As shown in FIGS. 15-18, the infusion set 200 can include a housing 204 configured to receive one or more components therein. The housing 204 may be similar to the body 14 of the infusion set 10 and/or the housing 104 of the infusion set adapter 100. The housing 204 includes an attachment surface 218 configured to contact the infusion site 40. A needle 222 can be disposed on, or in, the housing 204 and configured to be moved toward and into the infusion site 40. The needle 222 may be movable relative to the housing 204 or may be fixedly attached to the housing 204. The needle 222 may protrude, at least in part, from the attachment surface 218 toward the infusion site 40. The needle 222 has a needle tip 224 at an end thereof opposite the housing 204. A cannula 226 can extend into and/or through the housing 204. The cannula 226 can be connected to the medicament source 38. The attachment surface 218, the needle 222, and the cannula 226 may be similar to, or substantially the same as, the attachment surface 118, the needle 22, and the cannula 26, respectively, as described previously.

The infusion set 200 may include a needle actuator 230 configured to move the needle 222 between a sheathed configuration and an unsheathed configuration. The needle actuator 230 can include a button, a switch, or the like. The user can actuate the needle actuator 230 to cause the needle 222 to move from the sheathed configuration, in which the needle 222 is spaced from the infusion site 40, to the unsheathed configuration, in which the needle 222 is moved toward the infusion site 40. The needle 222 can contact the infusion site 40, and the needle tip 224 may pierce the infusion site 40.

The infusion set 200 can include a controller 250. The controller 250 can be similar to, or substantially the same as, the controller 150 described previously. One or more sensors may be disposed on or in the infusion set 200. The infusion set 200 can include any of the sensors described above with respect to the infusion set 10 and the infusion set adapter 100. In some particular aspects, the infusion set 200 can include a priming sensor 130, a proximity sensor 134, and a needle sensor 142. The priming sensor 130, the proximity sensor 134, and the needle sensor 142 may be substantially the same as those described with respect to the infusion set 10 and the infusion set adapter 100.

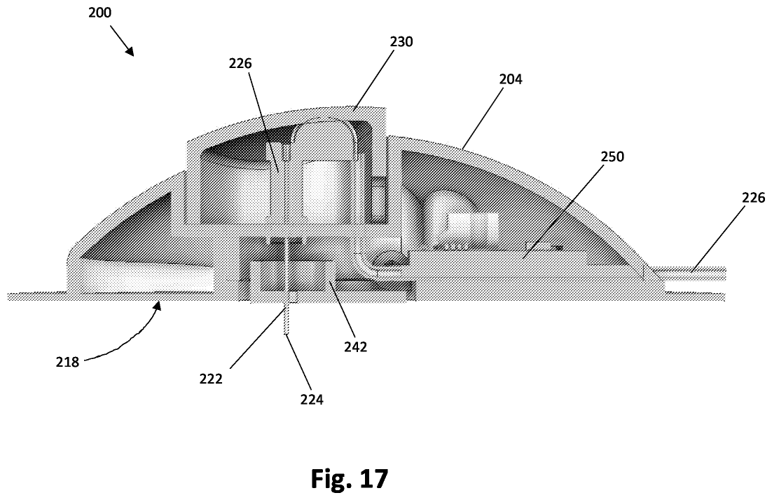

In some aspects, the infusion set 200 may include a needle guard 242 (see FIG. 17). The infusion set 200 may include a needle guard sensor 146 similar to ones described above. The needle guard 242 may be fixed to the housing 204 or may be movable relative to the housing 204.

Since the infusion set 200 integrates features of the infusion set 10 and the infusion set adapter 100, there is no need for a separate infusion set adapter 100 to be attached to the infusion set 200. As such, there may be no need for a connection sensor 138 in the infusion set 200 that can be used to determine if a proper connection exists between the infusion set 10 and the infusion set adapter 100. In some aspects, however, a connection sensor 138 may be disposed on the infusion set 200 and/or on the medicament source 38 that is configured to measure a characteristic associated with the connection between the medicament source 38 and the infusion set 200. The controller 250 can be configured to receive the measured characteristic from the connection sensor 138 and to determine if a suitable connection exists and/or if a suitable connection does not exist, as described previously.

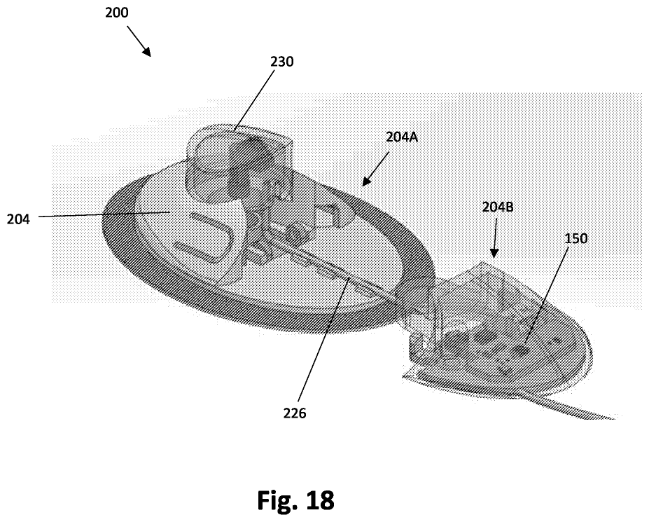

In some embodiments, some or all of the infusion set 200 can be designed to be reusable. It will be appreciated that proper sterilization and maintenance protocols should be followed when preparing and reusing medical devices. In some aspects, it may be preferable to reuse some, but not all, of the components of the infusion set 200, such as one or more of the sensors, controllers, or other electrical components. As shown in FIG. 18, for example, the housing 204 may include a disposable portion 204A and a reusable portion 204B that is separable from the disposable portion 204A. During use, the disposable portion 204A and the reusable portion 204B may be operably connected to one another. After use, the reusable portion 204B may be separated from the disposable portion 204A and sterilized according to known and accepted methods. The disposable portion 204A may be disposed of appropriately. The reusable portion 204B may include any one or more of the controller 250, the priming sensor 130, the proximity sensor 134, and the needle sensor 142. In some aspects, the infusion set 200 may include a connection sensor 138 configured to detect a parameter associated with the connection state of the disposable portion 204A with the reusable portion 204B. The controller 150 may be configured to receive information from the connection sensor 138 and to determine if the disposable and reusable portions 204A and 204B are connected or disconnected.

The disposable portion 204A may include one or more of the attachment surface 218, the needle 222, the needle actuator 230, and the needle guard 242. The cannula 26 can extend through, or adjacent to, one or both of the disposable portion 204A and the reusable portion 204B.

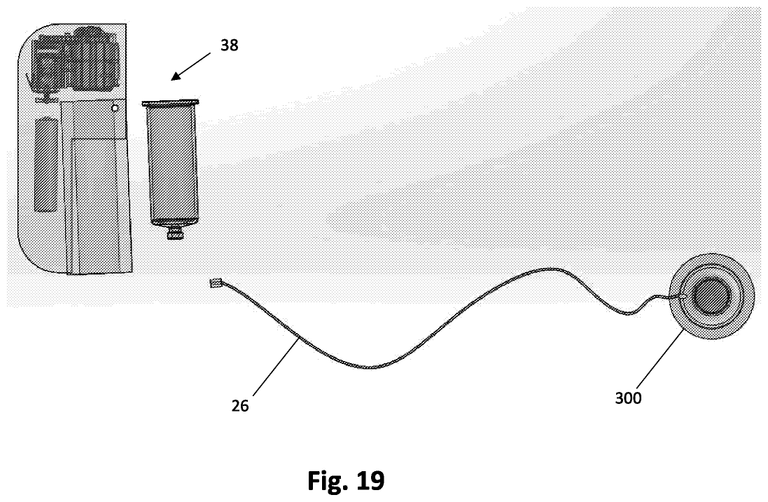

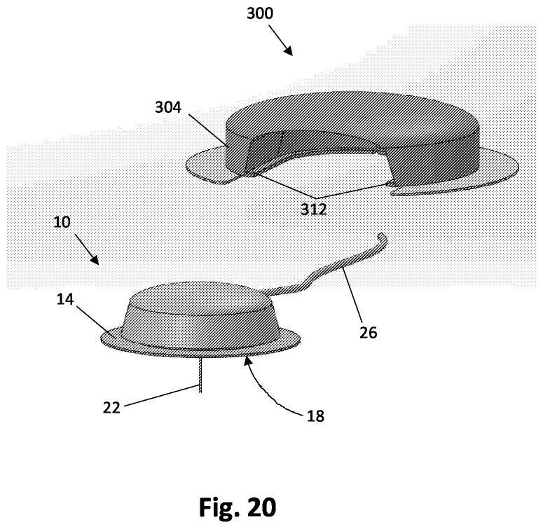

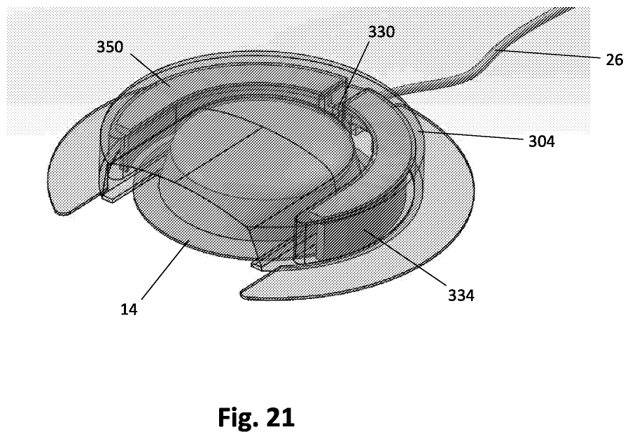

Referring to FIGS. 19-21, an infusion set adapter 300 is depicted according to another embodiment. Elements and components of the infusion set adapter 300 can be similar to, or substantially the same as, elements and components of the infusion set adapter 100 described previously. Descriptions of the features with respect to the infusion set adapter 100 can apply to similar features of the infusion set adapter 300. The infusion set adapter 300 has a housing 304 that is configured to attach to an infusion set, such as the infusion set 10 described previously or another suitable infusion set. The infusion set adapter 300 can include one or more retention members 312 configured to contact the infusion set 10 and to releasably secure the infusion set adapter 300 to the infusion set 10. The one or more retention members 312 can be similar to in structure and/or function to any of the retention members 112 described previously with respect to the infusion set adapter 100. As shown in FIG. 21, a controller 350 can be disposed on or in the housing 304. One or more sensors can be disposed on or in the housing 304 and be operably connected to the controller 350. For example, a priming sensor 330 may be disposed on the housing 304 and configured to detect a priming state of the cannula 26 connected to the infusion set 10. A proximity sensor 338 can be disposed on or in the housing 304 and configured to detect and measure the position of the infusion set adapter 300 and/or the infusion set 10 relative to the infusion site 40. The priming sensor 330 and the proximity sensor 334, as well as any other sensors, can be similar to, or substantially the same as, the priming sensor 130 and the proximity sensor 134, respectively, or another other sensors that were described with respect to the infusion set adapter 100.

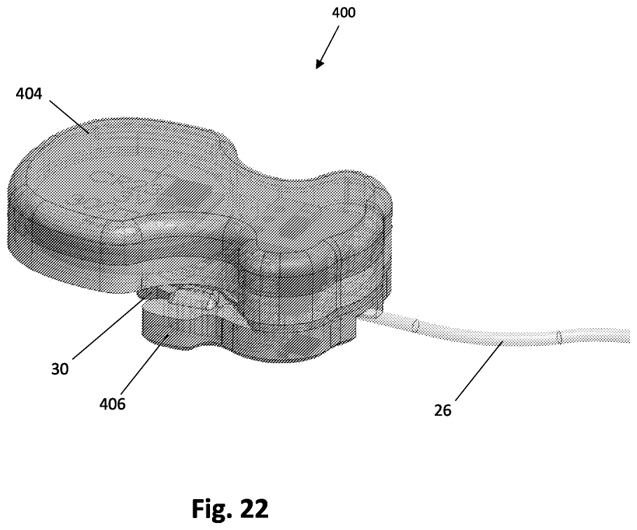

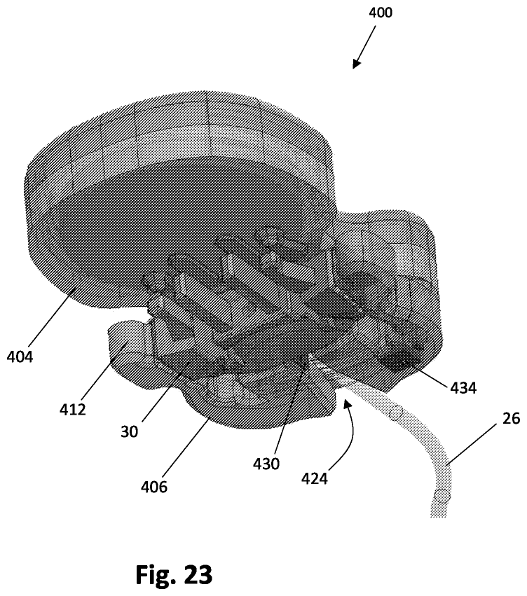

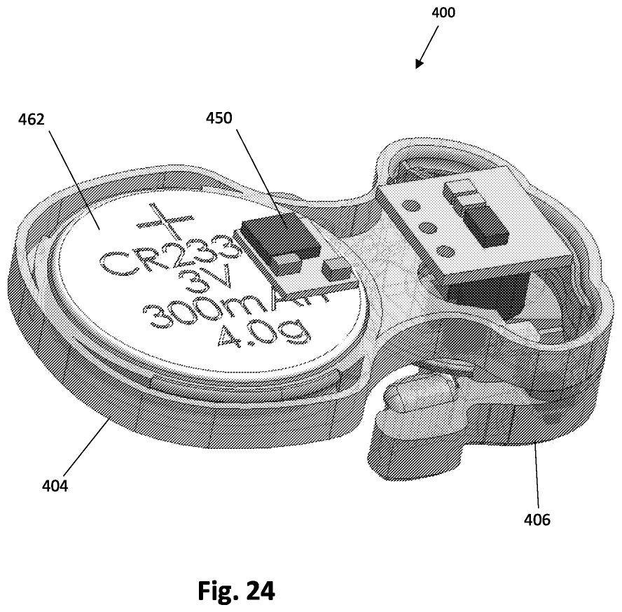

Referring to FIGS. 22-25, an infusion set adapter 400 is depicted according to yet another embodiment. A portion of an infusion set 10 is depicted relative to the infusion set adapter 400, showing the cannula 26 and the connector 30. Elements and components of the infusion set adapter 400 can be similar to, or substantially the same as, elements and components of the infusion set adapter 100 described previously. Descriptions of the features with respect to the infusion set 10 and/or the infusion set adapter 100 can apply to similar features of the infusion set adapter 400.

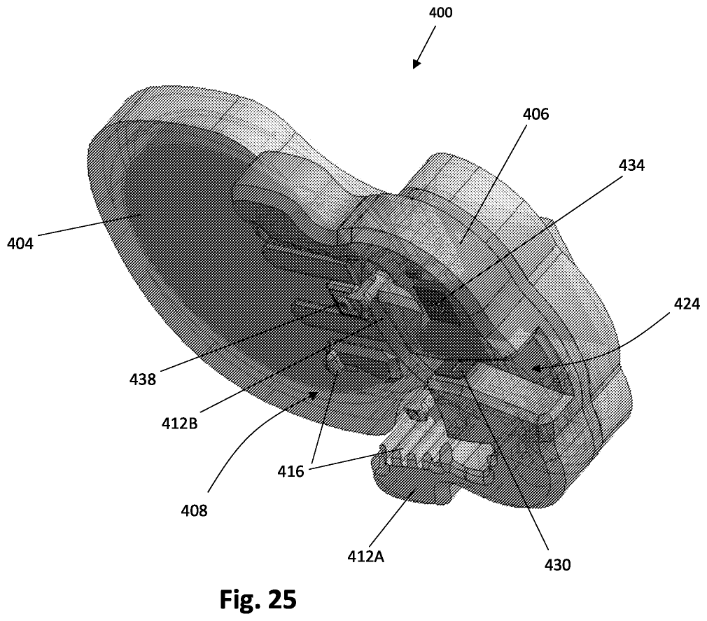

The infusion set adapter 400 has a housing 404 configured to connect to the infusion set 10 or to another suitable infusion set. The housing 404 can include an interface adapter 406. The housing 404 (e.g., the interface adapter 406) can define a receptacle 408 configured to receive a portion of the infusion set 10 therein when the infusion set adapter 400 is connected thereto. The housing 404 can have one or more retention members 412 (e.g., on the interface adapter 406). As shown in FIG. 25, the housing 404 can include a lateral retention member 412A and a vertical retention member 412B. The retention members 412 (e.g., lateral retention members 412A and vertical retention members 412B) can include one or more features 416 configured to engage with corresponding features on the connected infusion set 10 (e.g., with features 34 on the infusion set 10). The housing 404 can define a channel 424 extending therethrough configured to receive the cannula 26 therein. It should be understood that the housing 404 and retention members 412 can be similar to the housing 104 and retention members 112 described previously with respect to the infusion set adapter 100.

The infusion set adapter 400 can include a priming sensor 430 configured detect and measure a priming state of the cannula 26. The priming sensor 430 can be disposed adjacent to, or within, the channel 424. A proximity sensor 434 can be disposed on the housing 404 and be configured to detect the position of the infusion set adapter 400 and/or the infusion set 10 relative to the infusion site 40. The proximity sensor 434 can also measure the detected distance. A connection sensor 138 may be disposed on the housing 404 and configured to detect and measure a connection status between the infusion set adapter 400 and the infusion set 10. The priming sensor 430, the proximity sensor 434, and the connection sensor 438 can be similar to, or substantially the same as, the priming sensor 130, the proximity sensor 134, and the connection sensor 138 described previously. The infusion set adapter 400 can include a plurality of additional sensors, such as a needle sensor (not shown) or a needle guard sensor (not shown).

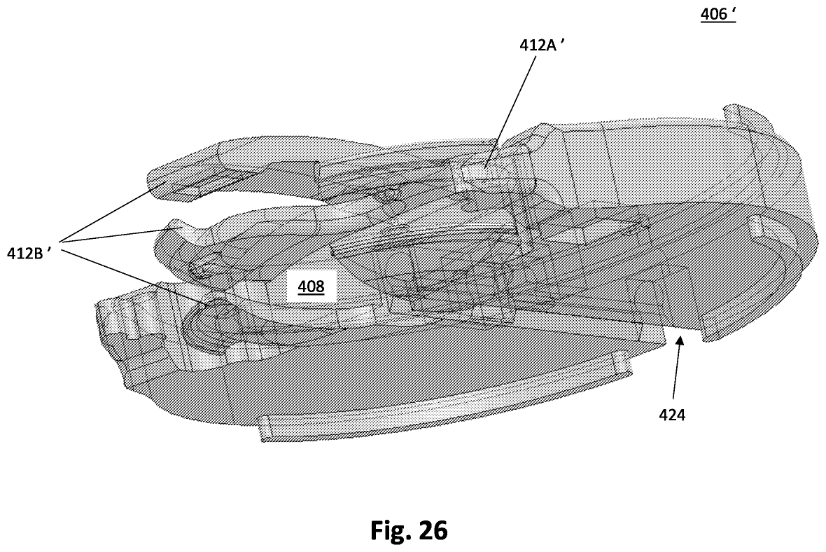

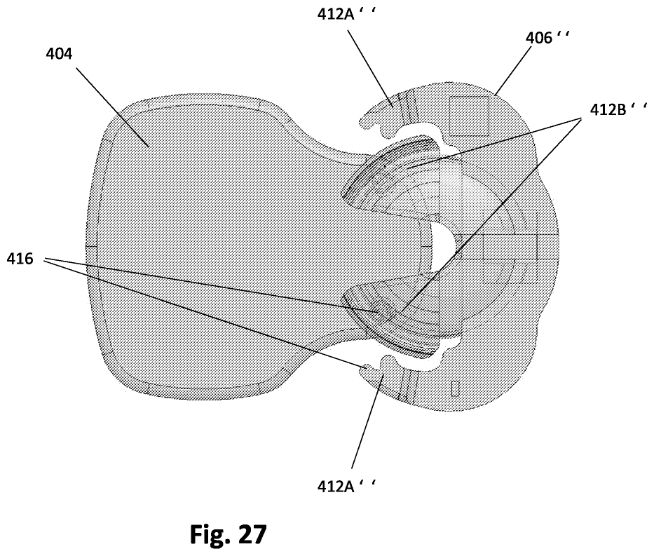

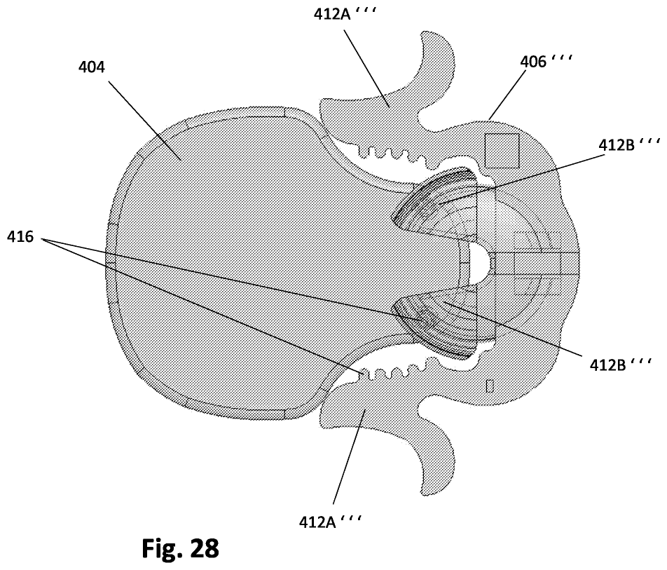

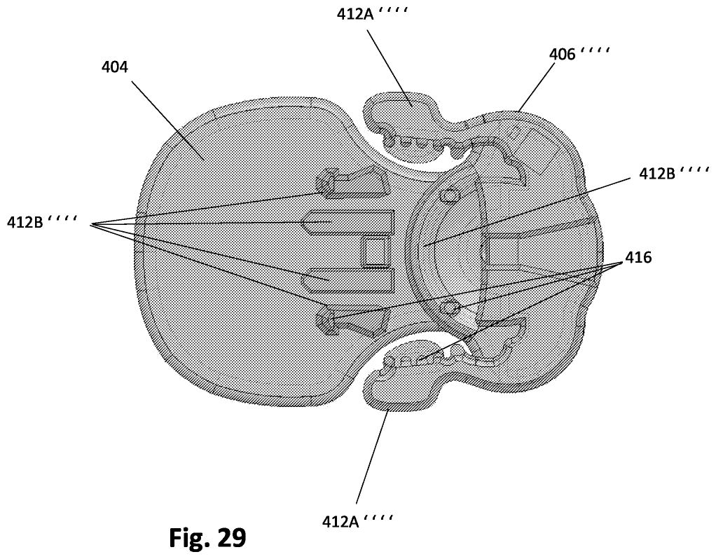

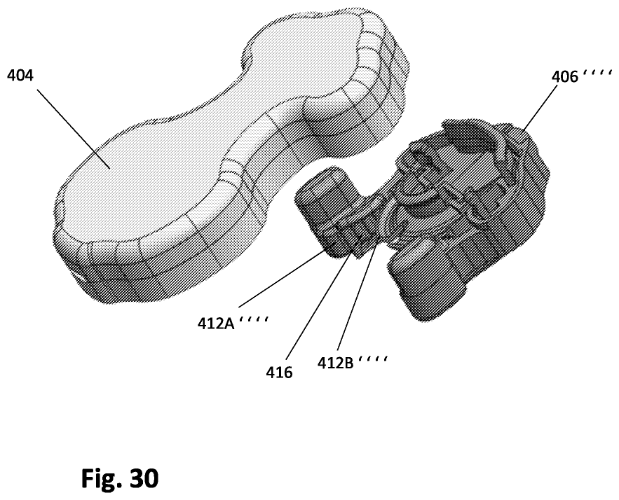

Different types of retention members 112, 312, and 412 can be utilized. It will be appreciated that the particular size, shape, orientation, and quantity of retention members can depend on the intended use of the infusion set adapter 100, 300, or 400 and on the infusion set 10 being connected. FIGS. 26-30 depict various embodiments of portions of housings 404 and interface adapters 406 having different retention members 412 thereon. It should be understood that described retention members 112 and 312 can also be designed to have similar or identical features as those shown in FIGS. 26-30. FIG. 26 depicts an interface adapter 406′ having lateral retention members 412A′ and vertical retention members 412B′. FIG. 27 depicts an interface adapter 406″ having lateral retention members 412A″ and vertical retention members 412B″. FIG. 28 depicts an interface adapter 406′″ having lateral retention members 412A′″ and vertical retention members 412B′″. FIGS. 29 and 30 depict an interface adapter 406″″ having lateral retention members 412A″″ and vertical retention members 412B″″. It will be appreciated that other suitable retention members 412 are envisioned.

Various components described throughout this application can be designed to be either single-use (i.e., disposable) or multi-use (i.e., reusable). In some aspects, the infusion set adapter 100 can be reusable, while the infusion set 10 may be disposable. In use, the disposable infusion set 10 can be attached to the infusion site 40, and the reusable infusion set adapter 100 can be attached to the infusion set 10. After use, the infusion set adapter 100 can be separated from the infusion set 10, and the infusion set 10 can be disposed of. The infusion set adapter 100 can be sterilized and prepared for re-use with another infusion set 10.

1. An infusion set adapter comprising:
a housing comprising a retention member configured to be connected to a body of an infusion set;
a controller and a power source, the power source configured to power the controller; and
a sensor in electrical communication with the controller, the sensor selected from the group consisting of a priming sensor adjacent a cannula of the infusion set configured to detect a presence of a medicament in the cannula, a proximity sensor configured to measure a characteristic of an infusion site, and a connection sensor configured to detect a characteristic indicative of a distance between the infusion set adapter and the infusion set.

2. The infusion set adapter of claim 1, wherein the sensor is the priming sensor and the controller is configured to cause a notification of the presence of the medicament in response to receiving a signal from the priming sensor.

3-5. (canceled)

6. The infusion set adapter of claim 1, wherein the retention member comprises a plurality of retention members configured to connect the housing to the infusion set.

7. The infusion set adapter of claim 1, wherein the retention member is resilient and configured to deform to move over a retention section of the infusion set as the retention member is being connected to the infusion set.

8. The infusion set adapter of claim 1, further comprising a needle configured to pierce into the infusion site.

9. The infusion set adapter of claim 8, further comprising a needle sensor configured to detect a characteristic of a needle tip of the needle within the infusion site, the needle sensor being in electrical communication with the controller.

10. The infusion set adapter of claim 8, further comprising a needle guard configured to cover a needle tip of the needle when the needle is not in the infusion site.

11. The infusion set adapter of claim 10, further comprising a needle guard sensor configured to detect a characteristic of the needle guard relative to the needle tip, the needle guard sensor being in electrical communication with the controller.

12. The infusion set adapter of claim 1, wherein the sensor is the proximity sensor and the controller is configured to determine if the infusion set adapter is within a predetermined distance from the infusion site.

13. (canceled)

14. The infusion set adapter of claim 12, wherein the proximity sensor includes:
an optical sensor configured to transmit a first optical signal to the infusion site and to receive a second optical signal reflected from the infusion site,
a temperature sensor configured to measure a temperature adjacent the proximity sensor, or
a mechanical sensor, an electromechanical sensor, or a pressure sensor configured to detect physical contact between the infusion set adapter and the infusion site.

15-16. (canceled)

17. The infusion set adapter of claim 1, wherein the sensor is the connection sensor and is disposed on the housing.

18. (canceled)

19. The infusion set adapter of claim 1, further comprising a removable interface adapter connected to the housing, the interface adapter having the retention member thereon.

20. An infusion set for delivering a medicament from a medicament source to an infusion site, the infusion set comprising:
a housing configured to be connected to the medicament source, the housing having an attachment surface for contacting the infusion site;
a cannula connected to the housing, the cannula being configured to receive the medicament therein from the medicament source;
a needle disposed on the housing, the needle comprising a needle tip configured to pierce the infusion site;
a needle guard configured to cover the needle tip when the needle tip is not in the infusion site;
a controller and a power source configured to provide power to the controller; and
a sensor in electrical communication with the controller, the sensor selected from the group consisting of a priming sensor configured to detect a presence of the medicament in the cannula, a proximity sensor configured to measure a characteristic of the infusion site, a connection sensor configured to detect a characteristic indicative of a connection between the medicament source and the infusion set, a needle sensor configured to measure a characteristic of the needle, and a needle guard sensor configured to measure a characteristic of the needle guard.

21. The infusion set of claim 20, wherein the controller is configured to cause a notification of the presence of the medicament in response to receiving a signal from the priming sensor.

22. The infusion set of claim 20, wherein the priming sensor is disposed within a lumen defined by the cannula or adjacent an outer surface of the cannula.

23-26. (canceled)

27. The infusion set of claim 20, further comprising adhesive on the attachment surface, the adhesive being configured to secure the infusion set to the infusion site.

28. The infusion set of claim 20, wherein the sensor is the proximity sensor, and the controller is configured to determine if the infusion set is within a predetermined distance from the infusion site.

29. (canceled)

30. The infusion set of claim 20, wherein the needle is configured to pierce the infusion site to create an aperture in the infusion site, and wherein the cannula is configured to be moved into the infusion site through the aperture.

31. An infusion set for delivering a medicament from a medicament source to an infusion site, the infusion set comprising:
a body configured to attach to the infusion site;
a needle extending from the body, the needle being configured to pierce the infusion site;
a cannula connected to the body, the cannula configured to receive the medicament from the medicament source therein; and
the infusion set adapter of claim 1.

32. The infusion set of claim 31, wherein the infusion set adapter is configured to be separated from the infusion set and sterilizable.

33-46. (canceled)
